(19) United States
(12) Patent Application Publication
(10) Pub. No.: US 2017/0128564 A1
(43) Pub. Date: May 11, 2017

(54) POLYPEPTIDE DERIVED FROM gp41, A VACCINE COMPOSITION COMPRISING SAID POLYPEPTIDE, AND USES FOR TREATING AN INFECTION BY AN HIV VIRUS IN AN INDIVIDUAL

(71) Applicants: INSTITUT NATIONAL DE LA SANTE ET DE LA RECHERCHE MEDICALE (INSERM), Paris Cedex 13 (FR); ASSISTANCE PUBLIQUE HOPITAUX DE PARIS, Paris (FR)

(72) Inventors: Vincent VIEILLARD, Paris (FR); Patrice DEBRE, Paris (FR)

(21) Appl. No.: 15/360,175

(22) Filed: Nov. 23, 2016

Related U.S. Application Data
(63) Continuation of application No. 14/286,467, filed on May 23, 2014, now abandoned.
Application No. 14/286,467 is a continuation-in-part of application No. 12/362,124, filed on Jan. 29, 2009, now abandoned.
Application No. 12/362,124 is a continuation of application No. 10/588,633, filed on Aug. 4, 2006, now abandoned.
Application No. 10/588,633 is a continuation of application No. PCT/EP2004/001106, filed on Feb. 6, 2004.

(30) Foreign Application Priority Data
Feb. 6, 2003 (EP) 03290303.1

(51) Int. Cl. A61K 39/21; C07K 16/10

(52) U.S. Cl. CPC A61K 39/21; C07K 16/1063; A61K 2039/6081

(57) ABSTRACT

The present invention relates to the field of the in vitro diagnosis of the progression status of an infection of an individual with a virus belonging to the family of the Human Immunodeficiency Viruses (HIV) as well as with the therapeutical treatment of this infectious disease. The invention also relates to immunological compounds and vaccine compositions comprising a polypeptide derived from gp41.

### CROSS-REFERENCE TO RELATED APPLICATIONS

This application is a continuation from U.S. Ser. No. 14/286,467 filed May 23, 2014, which is a continuation-in-part from U.S. Ser. No. 12/362,124 filed Jan. 29, 2009, which is a continuation from U.S. Ser. No. 10/588,633 filed Aug. 4, 2006, which is a 35 U.S.C. §371 application of PCT/EP2005/001395 filed Feb. 7, 2005, which is a continuation from PCT/EP2004/001106 filed Feb. 6, 2004, which claims priority to EP 03290303.1 filed Feb. 6, 2003. The entire text of each of the above-referenced disclosures is specifically incorporated herein by reference without disclaimer.

### FIELD OF THE INVENTION

The present invention relates to the field of the in vitro diagnosis of the progression status of an infection of an individual with a virus belonging to the family of the Human Immunodeficiency Viruses (HIV) as well as with the therapeutical treatment of this infectious disease.

### BACKGROUND OF THE INVENTION

AIDS disease, which is primarily caused by infection of individuals with a HIV retrovirus, is now the most devastating disease in the whole world, since the number of individuals which are, to date, infected with HIV viruses is estimated to about 40 millions of individuals.

During the sole year 2001, 5 millions of individuals were infected with HIV while 3 millions of individuals have deceased in the same time. Since the discovery of the main AIDS causative agent in 1983, namely the HIV virus, extensive efforts have been made in order to understand the mechanism of action of this virus and to develop accurate methods for (i) reproducibly diagnosing the infection, as well as (ii) carrying out a prognosis of the progression of the disease in a given patient.

For surveillance purposes, the United States Centers for Disease Control (CDC) currently defines AIDS in an adult or adolescent age 13 years or older as the presence of one of 25 AIDS-indicator conditions, such as KS, PCP or disseminated MAC. In children younger than 13 years, the definition of AIDS is similar to that in adolescents and adults, except that lymphoid interstitial pneumonitis and recurrent bacterial infections are included in the list of AIDS-defining conditions (CDC, 1987b). The case definition in adults and adolescents was expanded in 1993 to include HIV infection in an individual with a CD4⁺ T cell count less than 200 cells per cubic millimeter (mm³) of blood (CDC, 1992). The current surveillance definition replaced criteria published in 1987 that were based on clinical conditions and evidence of HIV infection but not on CD4⁺ T cell determinations (CDC, 1987).

In clinical practice, symptomatology and measurements of immune function, notably levels of CD4⁺ T lymphocytes, are used to guide the treatment of HIV-infected persons.

HIV infects and kills CD4⁺ T lymphocytes in vitro, although scientists have developed immortalized T-cell lines in order to propagate HIV in the laboratory (Popovic et al., 1984; Zagury et al., 1986; Garry, 1989; Clark et al., 1991). Several mechanisms of CD4⁺ T cell killing have been observed in lentivirus systems in vitro and may explain the progressive loss of these cells in HIV-infected individuals (reviewed in Garry, 1989; Fauci, 1993a; Pantaleo et al., 1993a). These mechanisms include disruption of the cell membrane as HIV buds from the surface (Leonard et al., 1988) or the intracellular accumulation of heterodisperse RNAs and unintegrated DNA (Pauza et al., 1990; Koga et al., 1988). Evidence also suggests that intracellular complexing of CD4 and viral envelope products can result in cell killing (Hoxie et al., 1986).

In addition to these direct mechanisms of CD4⁺ T cell depletion, indirect mechanisms may result in the death of uninfected CD4⁺ T cells (reviewed in Fauci, 1993a; Pantaleo et al., 1993a). Uninfected cells often fuse with infected cells, resulting in giant cells called syncytia that have been associated with the cytopathic effect of HIV in vitro (Sodroski et al., 1986; Lifson et al., 1986). Uninfected cells also may be killed when free gp120, the envelope protein of HIV, binds to their surfaces, marking them for destruction by antibody-dependent cellular cytotoxicity responses (Lyerly et al., 1987). Other autoimmune phenomena may also contribute to CD4⁺ T cell death since HIV envelope proteins share some degree of homology with certain major histocompatibility complex type II (MHC-II) molecules (Golding et al., 1989; Koenig et al., 1988).

A number of investigators have suggested that superantigens, either encoded by HIV or derived from unrelated agents, may trigger massive stimulation and expansion of CD4⁺ T cells, ultimately leading to depletion or anergy of these cells (Janeway, 1991; Hugin et al., 1991). The untimely induction of a form of programmed cell death called apoptosis has been proposed as an additional mechanism for CD4+ T cell loss in HIV infection (Ameisen and Capron, 1991; Terai et al., 1991; Laurent-Crawford et al., 1991). Recent reports indicate that apoptosis occurs to a greater extent in HIV-infected individuals than in non-infected persons, both in the peripheral blood and lymph nodes (Finkel et al., 1995; Pantaleo and Fauci, 1995b; Muro-Cacho et al., 1995).

It has also been observed that HIV infects precursors of CD4⁺ T cells in the bone marrow and thymus and damages the microenvironment of these organs necessary for the optimal sustenance and maturation of progenitor cells (Schnittman et al., 1990b; Stanley et al., 1992). These findings may help explain the lack of regeneration of the CD4+ T cell pool in patients with AIDS (Fauci, 1993a).

Recent studies have demonstrated a substantial viral burden and active viral replication in both the peripheral blood and lymphoid tissues even early in HIV infection (Fox et al., 1989; Coombs et al., 1989; Ho et al., 1989; Michael et al., 1992; Bagnarelli et al., 1992; Pantaleo et al., 1993b; Embretson et al., 1993; Piatak et al., 1993). One group has reported that 25 percent of CD4⁺ T cells in the lymph nodes of HIV-infected individuals harbor HIV DNA early in the course of disease (Embretson et al., 1993). Other data suggest that HIV infection is sustained by a dynamic process involving continuous rounds of new viral infection and the destruction and replacement of over 1 billion CD4⁺ T cells per day (Wei et al., 1995; Ho et al., 1995).

Concerning the prognosis of progression of the disease in HIV-infected patients, a first current method consists of evaluating the increase in the number of HIV viruses which are present in a whole blood sample collected from a patient, for example by performing conventional immunoassays with antibodies specifically directed against HIV proteins, and more specifically against the HIV capsid glycoprotein gp120.

A second current method for the prognosis of progression of AIDS in a patient consists of measuring the number of copies of the HIV genome which is found in a whole blood sample collected from that patient, for example through performing a quantitative PCR amplification of the nucleic acids contained in said sample, using one or several nucleic acid primer(s) that specifically hybridise with the HIV genomic RNA.

These two methods above are useful, since numerous studies have shown that people with high levels of HIV in their blood stream are more likely to develop new AIDS-related symptom or die than individuals with lower levels of the virus.

A third current method for the prognosis of progression of AIDS in a patient consists of measuring the absolute CD4⁺ T-cell levels in whole blood samples from infected patients (HIV⁺ patients), for example by carrying out flow cytometry from a blood sample of that patient, using a labelled antibody directed against the CD4 antigen.

All of these prognosis methods above can reproducibly be used but also have their respective technical limits, in relation with, for example, their biological significance as regards the evolution of the disease.

The use of antibodies for evaluating the number of HIV viral particles present in a biological sample form a patient comprise drawbacks due to the specificity of the antibodies which are used, since it is well known that the HIV structural proteins produced by distinct HIV virus isolates significantly differ in their antigenic properties and that false negative results may thus be generated.

The measure of the number of copies of the HIV genome in a biological sample from a patient is indeed indicative that the provirus which has integrated within the infected individual's cell genome has entered into active replication cycles and that the disease is in active progression. However, this technique does not simultaneously reflect the patient's immune response against the virus progression.

The measure of the CD4⁺ T-cell levels in a patient is also indicative of the disease progression, since the pathogenesis of acquired immunodeficiency syndrome (AIDS) is largely attributable to the decrease in T-lymphocytes bearing the CD4 receptor (CD4⁺). Progressive depletion of CD4⁺ T-lymphocytes is associated with an increase of clinical complications. Because of this association, the measurement of CD4⁺ T-cell levels is used to establish decision points for monitoring the relevance of treatments against AIDS. CD4⁺ T-lymphocyte levels are also used as prognostic indicators in patients with human immunodeficiency virus (HIV) disease.

However, the measure of the CD4⁺ T-cell levels in a patient does not directly reflect the immunological status of the patient, excepted as regards the resulting immunodeficiency. Notably the measure of the CD4⁺ T-cell levels does not account for the status of the possible biological effectors that cause or mediate the observed CD4⁺ depletion, and thus of the possible biological effectors that cause this observed patient's immunodeficiency.

Indeed, it may also be mentioned that a forecast of the progression of AIDS, in a given patient infected with HIV, can also be carried out through the detection of mutations occurring in the amino acid sequence of known co-receptors for HIV that are expressed by the patient's cells, especially CD4⁺ cells, such as the CCR5 co-receptor, since it has been observed that HIV-infected people bearing a specific mutation in one of their two copies encoding the CCR5 co-receptor may have a slower disease course that people with two normal copies of this gene.

However, there remains a need in the art for additional methods that will allow the one skilled in the art to determine the status of progression of AIDS in patients who have been infected with a HIV virus so as to enable a more precise prognosis of the evolution of the disease, including the occurrence of, or the evolution of, the numerous well known AIDS-related diseases, and also to enable a more precise monitoring of the therapeutical treatment which may be the more beneficial to the HIV-infected patient, once taken into account the progression status of the AIDS disease. For example, there is a need in the art for novel biological markers which are indicative of the progression of AIDS, which should preferably be of biological relevance as regards the biology of the HIV infection, such as, for example, novel biological markers of relevance as regards the immunological status of the patient tested.

Indeed, these novel biological markers might be used in combination with one or several already known markers such as those cited above.

Further, there is still a need in the art for novel therapeutically useful compounds for preventing individuals from the occurrence of AIDS upon infection with a HIV virus or, more generally, for treating patients infected with a HIV virus. Particularly, in the definition of novel anti-HIV multi-therapies or HAART (“Highly Active Anti-retroviral Therapy”), there is a need to include novel pharmaceutically active compounds that will specifically be directed against other target molecules than the HIV protease and the HIV retrotranscriptase and which will act on targets involved in distinct stages of the disease. Notably, there is a need in the art for novel compounds of pharmaceutical interest that are biologically active in HIV-infected patients wherein HIV has begun to actively replicate, especially in HIV-infected patient which are close to undergo a decrease in the number of their CD4⁺ T-cells and who are thus susceptible to immunodeficiency, as well as in HIV-infected patients for whom the depletion of their CD4⁺ T-cells has already begun.

### SUMMARY OF THE INVENTION

The invention is firstly directed to a polypeptide comprising the following amino acid sequence:

X₁X₂X₃X₄X₅X₆SWSNKSX₇X₈X₉X₁₀X₁₁ (I),

wherein X₁, X₂, X₃, X₅, X₆, X₇, X₉, X₁₀, and X₁₁ mean, independently one from each other, any amino acid residue, X₄ means any amino acid residue except A and W, and wherein X₈ means any amino acid residue except E and S.

It also relates to a pharmaceutical composition for preventing or treating a disease linked to the infection of an individual with a virus of the HIV family, which comprises an effective amount of a ligand compound which specifically binds to the polypeptide (I) in combination with at least one physiologically acceptable excipient.

The invention also deals with in vitro methods for the screening of compounds for preventing or treating a disease linked with the infection of an individual with an HIV virus, wherein said method comprises the steps of:
(i) incubating a candidate compound to be tested with a polypeptide as described above,
(ii) assaying for the binding of the candidate compound to be tested with a polypeptide as described above.

The invention is also directed to a vaccine composition comprising a polypeptide (I) and an immunoadjuvant compound.

It is also directed to methods for treating HIV-infected patients that make use of the therapeutically active compounds and of the pharmaceutical compositions that are further described in the present specification.

### DESCRIPTION OF THE FIGURES

FIG. 1. Over-expression of NKp44L after treatment of purified CD4+ T cells with vaccinia virus expressing several HIV proteins. Purified CD4+ T cells were infected with 20 pfu/cell of several recombinant vaccinia virus expressing HIV protein. Two days later, the cells were washed twice, and stained with anti-NKp44L mAb (grey thick line), or with IgM isotype control (blak thin line). The cells were analyzed by flow cytometry. UI: Uninfected cells, WT: cells infected with wild type vaccinia virus. Gag, Pol, gp160, gp120, gp41, Tat, Nef: cells infected with vaccinia virus, expressing respectively Gag, Pol, gp160, gp120, gp41, Tat, or Nef. The percentage of NKp44L expression was noted for each panel. Abscissa: NKp44L expression, Ordinates: Number of cells.

FIG. 2A-2B. Over-expression of NKp44L after treatment of purified CD4+ T cells with recombinant gp160 HIV protein. One million of cells were incubated with 5 ug/ml of control protein (Ctl; black circle), or recombinant gp160 protein (gp160-A: black triangle); (gp160-B: black square) or without protein (UT: untreated cells) during 2 days in presence of 10 U/ml IL2.

2A) The cells were washed and stained with anti-NKp44L mAb and CD4 mAb or with isotype controls and analyzed by flow cytometry. The percentage of NKp44L expression in CD4+ T cells was noted for each panel. Abscissa: CD4 expression, Ordinates: NKp44L expression.

2B) NK-lysis sensitivity of CD4+ T cells incubated with recombinant gp160 HIV protein was analyzed for cytotoxic activity with activated autologous purified NK cells. NK lysis activity was performed at different effetctor/target (E/T) ratios (Abscissa). Open diamonds with dotted lines: Unteated cells; Closed bottoms: Control protein-treated cells; Closed triangles: gp160-A-treated cells; and closed squares: gp160-B-treated cells. Ordinates: Specific NK lysis (%).

FIG. 3. One pool of peptides from the HIV gp41 protein both induced an higher sensitivity to NK lysis and an over-expression of NKp44L. One million of purified CD4+ T cells were treated with 5 μg/ml of pools of peptides from HIV gp41 protein (noted from A to J) or from gp120 protein (gp120), as control. Each pool of peptides included 10 peptides, as described in Material and Methods section. The cells were incubated two days in presence of 10 μ/ml IL2, and then washed twice.

A) NK-lysis sensitivity of CD4+ T cells incubated with the different pools of peptides was analyzed for cytotoxic activity with activated autologous purified NK cells. NK lysis activity was performed at different effector/target (E/T) ratios (Abscissa). Ordinates: Specific NK lysis (%).

B) The cells were stained with anti-NKp44L mAb and CD4 mAb or with isotype controls and analyzed by flow cytometry. In this panel of figures, the results were only done for the untreated cells (none) or the cells treated with polls of peptides from gp120 or from the gp41 (polls C and J). The percentage of NKp44L expression in CD4+ T cells was noted for each panel. For the other pools a low expression of NKp44L, ranged from 0.2 to 1.3%, was observed. Abscissa: NKp44L expression, Ordinates: CD4 expression.

FIG. 4. Analysis of each peptide from the pool C derived from HIV gp41 protein. One million of purified CD4+ T cells were treated with 5 μg/ml of peptides from the pool C (see FIG. 6) (noted from C141 to C150) or, as controls, the peptide gp41-E162 or the peptide gp120-87. The cells were incubated two days in presence of 10 μ/ml IL2, and then washed twice.

A) Killing pattern of CD4+ T cells incubated with the different peptides were tested for their sensitivity to NK. Data are shown for an E/T ratio of 40/1 with activated autologous purified NK effector cells. Ordinates: Specific NK lysis (%).

B) The cells were stained with anti-NKp44L mAb and CD4 mAb or with isotype controls and analyzed by flow cytometry. Ordinates: Expression of NKp44L.

FIG. 5. Drastic role of the NH2-SWSNKS-COOH motif expressed by the gp41 HIV protein.

A) Sequences of the peptide gp41-C147 (wild type: WT) and two different control peptides included some modification just inside the “SWSNKS” motif (control 1: Ctl1) or in all of the 15-mers sequence (control 2: Ctl2). One million of purified CD4+ T cells were treated with 1 ug/ml of highly purified WT peptide or with the both control peptides (Ctl1 and Ctl2). The cells were incubated two days in presence of 10 μ/ml IL2, and then washed twice.

B) NK-lysis sensitivity of CD4+ T cells incubated with the different peptides was analyzed for cytotoxic activity with activated autologous purified NK cells. NK lysis activity was performed at different effector/target (E/T) ratios (Abscissa). Open diamonds with dotted lines: Untreated cells; Closed bottoms: WT peptide-treated cells; Closed squares: Ctl1-peptide-treated cells, and Closed triangles: Ctl2-peptide-treated cells. Ordinates: Specific NK lysis (%).

C) The cells were stained with anti-NKp44L mAb and CD4 mAb or with isotype controls and analyzed by flow cytometry. The percentage of NKp44L expression in CD4+ T cells was noted for each panel. Abscissa: NKp44L expression, Ordinates: CD4 expression.

FIG. 6. Kinetics studies of NK lysis activity and NKp44L expression after addition of the “active SWSNKS” peptide. One million of purified CD4+ T cells were treated with 1 μg/ml of highly purified wild type (WT) peptide or with the both control peptides (Ctl1 and Ctl2) during several times ranged from 0 to 2880 min. After incubation, the cells were washed twice and then analyzed for cytotoxic activity with activated autologous purified NK cells. NK lysis activity was performed at different effector/target (E/T) ratios 9A). NK cytotoxic activity was performed after pretreatment of cell with 10 μg/ml of anti-NK44L mAb (B). Flow cytometry analysis revealed the cell surface expression of NKp44L (A), and for the intra-cellular expression of NKp44L (B). Open diamonds with dotted lines: Untreated cells; Closed bottoms: WT peptide-treated cells; Closed squares: Ctl1-peptide-treated cells, and Closed triangles: Ctl2-peptide-treated cells; Open bottoms with dotted line: WT-peptide-treatment cells after pretreatment with anti-NKp44L mAb and Open squares with dotted line: Ctl1-peptide-treated cells after pretreatment with anti-Nkp44L mAb.

FIG. 7. Cell surface expression of NKp44L of different human cells. Cell surface expression of NKp44L of K562, Jurkat, and resting PBMC. The cells were incubated with 1 μg/ml of anti-NKp44L mAb anti-NKp44L mAb (grey thick line) or with the IgM isotype control (black thin), and analyzed by flow cytometry. Abscissa: NKp44L expression, Ordinates: number of cells.

FIG. 8A-8E. Identification of #7.1, an anti-NKp44L mAb that specifically inhibits NKp44-mediated NK lysis.

8A) Cell surface expression of NCR-Ig fusion proteins. Uninfected (UI) and HIV-1-infected (Sf2) U2 cells were incubated with 10 μg/ml of NKp30-Ig (30-Ig), NKp46-Ig (46-Ig) or NKp44-Ig (44-Ig) fusion proteins (black lines) or with the Ig control (dotted lines) and analyzed by flow cytometry. Frequency of NKp44-Ig expression is noted.

8B) Cell surface expression of NKp44L detected with #7.1 mAb. Uninfected (UI) and HIV-1-infected (Sf2) U2 cells were incubated with 1 μg/ml of #7.1 mAb (black line) or with the IgM isotype control (dotted line) and analyzed by flow cytometry.

8C) #7.1 mAb inhibits NKp44-Ig binding. HIV-1-infected U2 cells that had previously been stained with either 10!mg of NKp44-Ig fusion protein (lower panel; black line) or control NKp46-Ig fusion protein (upper panel; black line) were incubated with #7.1 mAb (gray line) or IgM isotype control (dotted line) for 1!h at 4° C. and washed twice in 1% PBS-BSA. The cells were then analyzed by flow cytometry. Percentage of cells expressing NKp44L, after NCR-Ig incubation, is noted.

8D) Inhibition of natural cytotoxicity by #7.1 mAb. Purified NK cells, cultured in the presence of 100 U/ml IL2, were analyzed for cytotoxic activity against either the uninfected U2 cells and the HIV-1-infected U2 cell line, after treatment with 10!mg/ml of #7.1 mAb, at different effector/target cell ratios. Open circles: IgM-isotype control-treated uninfected U2 cells; Closed circles: #7.1-treated uninfected U2 cells. Open squares: IgM-isotype control-treated HIV-1-infected U2 cells; Closed squares: #7.1-treated HIV-1-infected U2 cells.

8E) Inhibition of natural cytotoxicity by #44/8 anti-NKp44 mAb. Purified NK cells, cultured in presence of 100 U/ml IL2, were analyzed for cytotoxic activity against either the uninfected U2 cells or the HIV-1-infected U2 cell line, after treatment with 10 μg/ml of #44/8 anti-NKp44 mAb, at different effector/target cell ratios. Open circles: IgG1-isotype control-treated uninfected U2 cells; Closed circles: #44/8 anti-NKp44 mAb-treated uninfected U2 cells. Open squares: IgG1-isotype control-treated HIV-1 infected U2 cells; Closed squares: #44/8 anti-NKp44 mAb-treated HIV-1 infected U2 cells.

FIG. 9. Critical role of the NH2-SWSNKS-COOH motif from the gp41 HIV protein.

E) NKp44L effects were inhibited with anti-gp41-C146 polyclonal antibody. One million purified CD4+ T cells from two infected patients (#CG: 322 CD4+ cells/mm³ and #BT: 208 CD4+ cells/mm3) were treated overnight with several concentrations of anti-gp41-C146 antibody. The cells were stained with the anti-NKp44L mAb and analyzed by flow cytometry.

F) Inhibition of NK lysis activity in the presence of anti-gp41-C146 antibody. CD4+ T cells from #CG and #BT samples, incubated with several concentrations of anti-gp41-C146 antibody, were then analyzed for cytotoxic activity with IL2-activated autologous purified NK cells. Open circles: Untreated cells; Closed squares, triangles, and diamonds: CD4+ T cells treated with 1, 10, and 20 mg/ml of anti-gp41-C146 antibody, respectively. Peptide C146 is a polypeptide consisting of the amino acid sequence of formula (II).

### DETAILED DESCRIPTION OF THE INVENTION

### a) Previous Findings of the Inventors

It has previously been found by the inventors, that a specific protein, termed NKp44L is expressed by the CD4⁺ T-cells form HIV-infected individuals whereas this protein is not expressed by the CD4⁺ T-cells from individuals which are not infected with HIV. The NKp44L protein is not expressed (i) in peripheral blood mononuclear cells (PBMC) from HIV-infected patients that do not express the CD3 antigen, (ii) in PBMC form HIV-infected patients that express the CD3 antigen but not the CD4 antigen, nor (iii) in PBMC from HIV-infected patients expressing the CD8 antigen. Particularly, the expression level of the NKp44L protein is further enhanced in activated CD4⁺ T-cells, such as PHA-activated CD4⁺ T-cells, from HIV-infected individuals.

Further, it has been previously found by the inventors that an increasing expression level of the NKp44L protein is correlated with the decrease in the number of CD4⁺ T-cells which is observed in HIV-infected patients, thus in patients undergoing a progression of AIDS. Consequently, the expression level of the NKp44L protein is indicative of the immunological status of an HIV-infected patient.

It has also been found that CD4⁺ T-cells from HIV-infected patients, and especially CD4+ T-cells that express the NKp44L protein, consist of specific targets for their cytolysis by Natural Killer (NK) cells, particularly activated NK cells, and especially autologous NK cells from the same patient.

Importantly, the present inventors have shown that the NK cells of an HIV-infected individual are activated specifically, through a non-MHC dependent triggering mechanism, by the autologous CD4⁺ T-cells that express the NKp44L protein.

Further, the expression level of the NKp44L protein consists of a novel biological marker of the state of advancement of the HIV infection endowed with a very high biological significance, since it has been shown by the inventors that NKp44L expressed by the CD4⁺ T-cells triggers the autologous NK cells and activate these NK cells for specific cytolysis of the CD4⁺ T-cells, through a non-MHC dependent recognition of the CD4⁺ T-cells by the activated NK cells. In this particular context, the NKp44L protein expressed by the CD4⁺ T-cells of the HIV-infected patient activate the NK cells through the specific binding of the NKp44L protein to its specific receptor counterpart which is expressed at the membrane surface of the NK cells, namely the NKp44 receptor protein which has already been described by Cantoni et al. (1999) and by Vitale et al. (1998).

Further, the NKp44L protein has formerly been isolated by another inventive entity and this protein has already been shown to be expressed in various kinds of tumour cell lines. Still further, the NKp44L expressed by certain tumour cells has been shown to be a ligand that specifically binds to the NKp44 receptor protein cited above, which receptor protein is expressed by the NK cells, including the activated NK cells. It has also been formerly shown by this other inventive entity that the NKp44 receptor protein that is expressed by the activated NK cells might be responsible for at least part of the tumour cells cytolysis effected by the activated NK cells (unpublished information).

Taken together, the results obtained by the inventors have allowed them to carry out various methods which make use of the NKp44L protein as a novel biologically relevant marker of the disease progression for individuals that are already diagnosed as having been infected by HIV.

### b) Findings According to the Invention

The inventors have now surprisingly found that a specific polypeptide, derived from the gp41 protein from HIV, markedly enhances the expression of the Nkp44L protein of sequence SEQ ID No 1, at the membrane surface of CD4⁺ T-cells.

The NKp44L protein is encoded by a nucleic acid of sequence SEQ ID No 3.

It has also been determined according to the present invention that the lysis by the NK cells of the CD4+ T-cells from patients infected with HIV depends on that specific HIV polypeptide.

HIV-1 gp41 is composed of three domains, an extracellular domain (ectodomain), a transmembrane domain and an intracellular domain (endodomain). The gp41 ectodomain contains three major functional regions, i.e., the fusion peptide located at the N-terminus of gp41, followed by two 4-3 heptad repeats adjacent to the N- and C-terminal portions of the gp41 ectodomain, designated NHR (N-terminal heptad repeat) and CHR (C-terminal heptad repeat), respectively. The N- and C-terminal repeats are also named as “HR1” and “HR2”.

Both NHR and CHR regions function as essential structures required for conformational changes during the process of membrane fusion between HIV-1 and CD4⁺ T cells.

Surprisingly, the inventors have found that a short peptide, derived from the gp41 protein, which is located between the well-known HR1 and HR2 regions, induces the surface expression of NKp44L on CD4⁺ T cells.

In other words, the inventors have identified a short peptide derived from the gp41 protein of HIV, which is responsible for the NKp44L surface expression and thus also for the lysis of CD4+ T cells by the endogenous NK cells.

These results obtained by the inventors have allowed them to carry out screening methods, which make use of a specific peptide derived from gp41 as a new target for therapeutical agents, distinct from the well-known HR1 and HR2 regions.

Importantly, the present inventors have also shown that the protein NKp44L is expressed on tumor cell surface and that this expression of NKp44L is induced or enhanced by said short peptide derived from gp41.

Thus according to the invention, said short peptide derived from gp41 can be used for expressing NKp44L at the surface of tumor cells and then induce their specific lysis by NK cells.

Accordingly, the invention concerns therapeutical methods, and pharmaceutical compositions, comprising a polypeptide as briefly described above, for manufacturing anti-cancer pharmaceutical compositions.

In another aspect, the inventors have found that antibodies directed against a polypeptide derived from the gp41 protein, are produced during HIV infection of an individual.

More precisely, it has been found, according to the invention a statistically significant correlation between the expression level of antibodies directed against a polypeptide derived from gp41, collected from HIV-infected individuals and the level of CD4+ T cells.

It has also been found that these antibodies inhibit the cytotoxicity of NK cells towards CD4+ T cells, in patients infected by HIV.

Accordingly, the invention concerns therapeutical methods, and vaccine compositions, comprising a polypeptide as briefly described above.

It has also been found according to the invention that the level of the antibodies described above, decreases during the progression of HIV infection, especially as regards the development of the patient's immunodeficiency caused by the progressive depletion of his CD4+ T cells.

This result obtained by the inventors has allowed them to carry out methods which make use of the level of antibodies directed against a polypeptide derived from gp41, as a novel biologically relevant marker of the disease progression for individuals that are already diagnosed as having been infected by HIV.

### Polypeptides According to the Invention

Accordingly, an object of the invention is a polypeptide comprising the following amino acid sequence:

X₁X₂X₃X₄X₅X₆SWSNKSX₇X₈X₉X₁₀X₁₁ (I),

wherein X₁, X₂, X₃, X₅, X₆, X₇, X₉, X₁₀, and X₁₁ mean, independently one from each other, any amino acid residue, X₄ means any amino acid residue except A and W, and wherein X₈ means any amino acid residue except E and S.

The term “any amino acid residue” designates any amino acid residue selected from the group consisting of the following amino acids: A, R, N, D, C, Q, E, G, H, I, L, K, M, F, P, S, T, W, Y, and V.

Said polypeptide is also referred to as a polypeptide of formula (I).

The invention encompasses further polypeptides comprising the following amino acid sequence (SEQ ID No 5):

PWASNASWSNKSLDDIW (II).

In certain embodiments said polypeptide has a length of at least 17, 18, 19, 20, 25, 30, 35, 40, 45, 50, 60, 70, 80, 90, 100 or 200 amino acid residues.

A polypeptide, as defined above, is preferably derived from the gp41 protein and possesses at least 39, 40, 50, 60, 70, 80, 90, 100, 110, 120, 130, 140 or 150 consecutive amino acids of gp41 protein from HIV-1 and comprises the amino acid sequence of formula (I) above.

The polypeptide of formula (I) can be produced by recombinant DNA techniques, for example on the basis of the DNA sequence of gp41 protein from HIV1, by using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast, and higher eukaryotic cells, such as mammalian cells and plant cells. Preferably, the host cells employed are E. coli, yeast or a mammalian cell line such as COS or CHO. Supernatants from suitable host/vector systems which secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

When polypeptide of formula (I) comprises less than about 100 amino acids, and generally less than about 50 amino acids, it can be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

Preferably, a polypeptide of formula (I) consists of the following amino acid sequence (SEQ ID No 5):

PWASNASWSNKSLDDIW (II).

The induction of NKp44L expression on CD4+ T-cells surface induced by the polypeptide of amino acid sequence (II) is illustrated in examples 1-3 below.

The high kinetics of the induction of the NKp44L expression at the cell surface is compatible with the induction of a translocation of a pre-synthesised NKp44L intracellular protein, from the cytoplasm towards the cell surface.

### Pharmaceutical Compositions According to the Invention

Another object of the invention is a pharmaceutical composition for preventing or treating a disease linked to the infection of an individual with a virus of the HIV family, which comprises an effective amount of a ligand compound which specifically binds to the polypeptide of formula (I), in combination with at least one physiologically acceptable excipient.

By “physiologically acceptable excipient or carrier” is meant solid or liquid filler, diluent or substance which may be safely used in systemic or topical administration. Depending on the particular route of administration, a variety of pharmaceutically acceptable carriers well known in the art include solid or liquid fillers, diluents, hydrotropes, surface active agents, and encapsulating substances. The amount of carrier employed in conjunction with the F(ab).sub.2 fragments to provide practical quantity of material per unit dose of composition.

Pharmaceutically acceptable carriers for systemic administration that may be incorporated in the composition of the invention include sugar, starches, cellulose, vegetable oils, buffers, polyols and alginic acid. Specific pharmaceutically acceptable carriers are described in the following documents, all incorporated herein by reference: U.S. Pat. No. 4,401,663, Buckwalter et al. issued Aug. 30, 1983; European Patent Application No. 089710, LaHann et al. published Sep. 28, 1983; and European Patent Application No. 0068592, Buckwalter et al. published Jan. 5, 1983. Preferred carriers for parenteral administration include propylene glycol, pyrrolidone, ethyl oleate, aqueous ethanol, and combinations thereof.

Representative carriers include acacia, agar, alginates, hydroxyalkylcellulose, hydroxypropyl methylcellulose, carboxymethylcellulose, carboxymethylcellulose sodium, carrageenan, powdered cellulose, guar gum, cholesterol, gelatin, gum agar, gum arabic, gum karaya, gum ghatti, locust bean gum, octoxynol 9, oleyl alcohol, pectin, poly(acrylic acid) and its homologs, polyethylene glycol, polyvinyl alcohol, polyacrylamide, sodium lauryl sulfate, poly(ethylene oxide), polyvinylpyrrolidone, glycol monostearate, propylene glycol monostearate, xanthan gum, tragacanth, sorbitan esters, stearyl alcohol, starch and its modifications. Suitable ranges vary from about 0.5% to about 1%.

For formulating a pharmaceutical composition according to the invention, the one skilled in the art will advantageously refer to the last edition of the European pharmacopoeia or of the United States pharmacopoeia.

Preferably, the one skilled in the art will refer to the fourth edition “2002” of the European Pharmacopoeia, or also to the edition USP 25-NF20 of the United States Pharmacopoeia.

The weight amount of therapeutically active compound that is contained in each dose of the pharmaceutical composition of the invention will depend on the molecular weight of said therapeutically active compound as well as on the weight amount that is effective in blocking the cytolysis of the CD4+ T-cells by the NK cells in an HIV-infected patient.

For determining the appropriate amount of the therapeutically active compound, in a dose of a pharmaceutical composition of the invention, the one skilled in the art firstly determines the in vitro CD4⁺ T-cell cytolysis inhibiting ability of various weight amounts or concentrations of said therapeutically active compound, for example by performing the screening method of the invention which are describe below, and then retain or select the given amount or concentration of said therapeutically active compound that blocks cytolysis.

Then, the one skilled in the art transposes said retained or selected amount or concentration to the in vivo human situation, so that the concentration of said therapeutically active compound in the blood of a patient to which the pharmaceutical composition of the invention has been administered is identical to the concentration that blocks cytolysis in vitro.

Preferably, the ligand compound consists of an antibody directed to the polypeptide according to the invention.

Preferably, the ligand compound, or the pharmaceutical composition containing it, can be combined with a compound that inhibits the membrane fusion between HIV and CD4+ T cells. Such compounds are, for example, peptides derived from the HR1 or HR2 region of the gp41 protein and more precisely peptides referred to as T20, T21 or those described in U.S. Pat. No. 6,623,741.

The invention concerns also an antibody directed against a polypeptide of formula (I).

This invention is also directed to the use of a ligand compound which specifically binds to the polypeptide of formula (I), for manufacturing a pharmaceutical composition for preventing or treating a disease linked to the infection of an individual with a virus of the HIV family.

Additionally, the inventors have also shown that the protein NKp44L is expressed on tumor cell surface, such as Jurkat cells and K562 cells.

They have also shown that a polypeptide of formula (I) induces the cell surface expression, of NKp44L by tumor cells, which then render these polypeptide-treated tumor cells susceptible to specific lysis by the NK cells.

Accordingly, the invention concerns methods and pharmaceutical compositions, comprising a polypeptide of formula (I), for treating cancer.

The invention concerns a pharmaceutical composition for treating a cancer, which comprises an effective amount of an antigenic compound comprising or consisting of a polypeptide of formula (I), in combination with at least one physiologically acceptable excipient.

Preferably, the physiologically acceptable excipients used to carry out the pharmaceutical composition described above are the same than those that are described in the first part of the specification concerning ligands of NKp44L.

The invention concerns also a pharmaceutical composition for treating a cancer, which comprises an effective amount of an antigenic compound comprising or consisting of a polypeptide of formula (I), fused to a targeting cancer cells, in combination with at least one physiologically acceptable excipient.

Preferably, said compound, which targets cancer cells, consists of an antibody directed to an antigen specific of cancer, such as SCP-1, NY-ESO-1, or SSX-2 specific of breast cancer, SSX-2, NY-ESO-1, or MAGE-3 specific of melanoma, described in U.S. Pat. No. 6,338,947; or antigens specific of renal cancers such as those described in U.S. Pat. No. 6,440,663; KH-1 and N3 specific of colon cancer, described in U.S. Pat. No. 6,238,668.

### Immunogenic and Vaccine Compositions According to the Invention

The inventors have found that antibodies directed against a polypeptide derived from the gp41 protein, i.e. the polypeptide of formula (I), are produced during HIV infection of an individual.

More precisely, It has been found, according to the invention a statistically significant correlation between the expression level of antibodies directed the polypeptide of formula (I), collected from HIV-infected individuals and their level of CD4+ T cells.

It has also been found that these antibodies inhibit the cytotoxicity of NK cells towards CD4+ T cells, in patients infected by HIV.

Further, the inventors have surprisingly found that antibodies screened for their capacity to inhibit CD4+ T cells NK lysis, react specifically with NKp44L on HIV-1 infected cells (example 5).

More precisely, when cells chronically infected by HIV-1 are pre-treated with the antibody described above, their NK-mediated lysis decreased sharply (FIG. 9E), and treatment of NK cells by an anti-NKp44 mAb produced the same effect (FIG. 9F).

NKp44 is a protein having the amino acid sequence SEQ ID No 2, encoded by a nucleic acid of sequence SEQ ID No 4.

It has also been found that an antibody directed against a polypeptide of formula (I) above, inhibits NKp44L expression onto CD4+ T cells surface, and by the way, decreases sensitivity to NK lysis.

On the opposite, such a result is not obtained with antibodies directed against other polypeptides derived from gp41 protein, for example polypeptides T20 or T21, derived from HR1 or HR2 domains of gp41.

More precisely, it has been found that NKp44L expression was substantially lower in purified CD4+ T cells from two HIV-1 infected patients that were incubated with an antibody directed to the polynucleotide (I), than in purified and then untreated cells or in those treated with a control Ab. (example 6, FIG. 9)

It has also been found that the serum from patients infected by HIV, when depleted in antibodies directed against polypeptide (I) is not able to decrease the CD4+ T cells NK lysis.

These results strongly suggest that the polypeptide of formula (I) play a key role in inducing NKp44L expression during HIV infection and that the gp41 protein participates in the selective destruction of CD4+ T cells by activated NK cells.

Without wishing to be bound to any particular theory, the inventors believe that HIV-1 has acquired the ability to use NK cells to disarm the host immune system by selectively triggering CD4+ T cells.

Hence, the blockage of NKp44L expression by antibodies directed against polypeptide of formula (I) could be applied to counteract these deleterious effects.

To illustrate this hypothesis, the inventors have found that there exists an inverse relationship between NKp44L expression at the CD4+ T cell surface and the level of antibodies directed against the polypeptide derived from gp41, of formula (I).

Accordingly, another object of the invention is a vaccine composition comprising a polypeptide of formula (I) and an immunoadjuvant compound.

A further object of the invention is an immunogenic composition comprising a polypeptide of formula (I), in combination with at least one physiologically acceptable excipient.

By “immunogenic composition” it is herein intended a substance which is able to induce an immune response in an individual, and for example to induce the production of antibodies directed against the polypeptide of formula (I).

Preferably, said immunoadjuvant compound is selected in the group consisting of Freund complete adjuvant, Freund incomplete adjuvant, aluminium hydroxide, calcium phosphate, aluminium phosphate, potassium phosphate, Cholera toxin (CT) and its B subunit (CTB), toxins from Bordetella pertusssis (PT), labile toxin (LT) from Escherichia coli, monophosphoryl lipid A, CpG oligonucleotides, imidazoquinolones, oil in water emulsions, comprising squalene and synthetic copolymers, muramyl dipeptides and their derivatives, saponins and immunostimulating complexes (ISCOMs), and dimethyldioctadecylammonium bromide or chloride (DDA).

For example, to promote a Th2-type immune response, said immunoadjuvant can be selected in the group comprising: aluminium hydroxide, aluminium phosphate, potassium phosphate, calcium phosphate, or bacteria toxins such as Cholera toxin (CT) and its B subunit (CTB), toxins from Bordetella pertusssis (PT), or labile toxin (LT) from Escherichia coli. When a Th1-type response is searched, said immunoadjuant can be selected in the group comprising: monophosphoryl lipid A, CpG oligonucleotides, imidazoquinolones. To stimulate preferably an antibody response, oil-based adjuvants such as oil in water emulsions, comprising squalene and synthetic copolymers can be used as an immunoadjuvant or also muramyl dipeptides and their derivatives. To promote a mucosal immune response, polysaccharides such as dextran, mannans, glucans, or chitosans can be used as an immunoadjuvant. To enhance antibody production and stimulate T cytotoxic lymphocytes, saponins and immunostimulating complexes (ISCOMs) can be used as an immunoadjuvant. To induce a humoral and cellular response, a Freund complete adjuvant can be used as an immunoadjuvant. To induce an inflammatory response and a high level of antibodies, a Freund incomplete adjuvant can be used as an immunoadjuvant. To obtain a delayed hypersensitivity lipophilic amines such as dimethyldioctadecylammonium bromide or chloride (DDA) can be used as an immunoadjuvant.

In order to enhance the immunogenicity of the vaccine composition according to the invention, the polypeptide of formula (I), can comprise from 2 to 12 peptides of formula “SWSNKS” (SEQ ID No 9).

In particular, said antigenic polypeptide, can have the following formula (III):

NH₂-PepNt-[(I)ₙ-PepXₙ]ₙ-PepCt-COOH (III),

wherein:
“PepNt” consists of a polypeptide having an amino acid length varying from 0 to 100 amino acid residues and located at the N-terminal end of the polypeptide of formula (III);
“[(I)ₙ-PepXₙ]” consists of a polypeptide unit wherein:
   “(I)₁” to −“(I)ₙ” each consists of, one independently from each other, a polypeptide of formula “SWSNKS” (SEQ ID No 9), with n being an integer from 1 to 12; and
   “PepX₁” to “PepXₙ” each consists of, one independently from the other, a spacer polypeptide having an amino acid length varying from 0 to 30 amino acid residues, with n being an integer from 1 to 12;
n is the number of [(I)ₙ-PepXₙ] polypeptide units in said polypeptide, with n being an integer from 1 to 12; and
“PepCt” consists of a polypeptide having an amino acid length varying from 0 to 100 amino acid residues and located at the C-terminal end of the polypeptide of formula (III).

The present invention also relates to an antigenic peptide of formula (III) as described herein, to immunogenic or vaccine compositions comprising the said antigenic peptide of formula (III), as well as to methods for treating an HIV-infected patient comprising a step of administering, to a patient in need thereof, the said antigenic peptide or an immunogenic or a vaccine composition comprising the said antigenic peptide.

In a preferred embodiment, the said antigenic polypeptide is represented by a polypeptide of the following formula (III):

NH₂-PepNt-[(I)ₙ-PepXₙ]ₙ-PepCt-COOH (III),

wherein:
“PepNt” consists of a polypeptide having an amino acid length varying from 2 to 10 amino acid residues and located at the N-terminal end of the polypeptide of formula (III);
“[(I)ₙ-PepXₙ]” consists of a polypeptide unit wherein:
   “(I)₁” to −“(I)ₙ” each consists of, one independently from each other, a polypeptide of formula “SWSNKS” (SEQ ID No 9), with n being an integer of 1; and
   “PepX₁” to “PepXₙ” each consists of, one independently from the other, a spacer polypeptide having an amino acid length of 0 amino acid residue, with n being an integer of from 1 to 12; which encompasses n being 1,
n is the number of [(I)ₙ-PepXₙ] polypeptide units in said polypeptide, with n being an integer of from 1 to 12; which encompasses n being 1, and
“PepCt” consists of a polypeptide having an amino acid length varying from 2 to 10 amino acid residues and located at the C-terminal end of the polypeptide of formula (III).

In these embodiments wherein “PepX₁” to “PepXₙ” each consists of, one independently from the other, a spacer polypeptide having an amino acid length of 0 amino acid residue, “[(I)ₙ-PepXₙ]” may also be represented by the formula “[(I)ₙ]” and the polypeptide of formula (III) may also be represented by the following formula (VII):

NH₂-PepNt-[(I)ₙ]ₙ-PepCt-COOH (VII),

with the variable parameters as defined above.

In a still preferred embodiment, the said antigenic polypeptide is represented by a polypeptide of the following formula (III):

NH₂-PepNt-[(I)ₙ-PepXₙ]ₙ-PepCt-COOH (III),

wherein:
“PepNt” consists of a polypeptide having an amino acid length of 4 amino acid residues and located at the N-terminal end of the polypeptide of formula (III);
“[(I)ₙ-PepXₙ]” consists of a polypeptide unit wherein:
   “(I)₁” to −“(I)ₙ” each consists of, one independently from each other, a polypeptide of formula “SWSNKS” (SEQ ID No 9), with n being an integer of 1; and
   “PepX₁” to “PepXₙ” each consists of, one independently from the other, a spacer polypeptide having an amino acid length of 0 amino acid residues, with n being an integer of from 1 to 12; which encompasses n being 1,
n is the number of [(I)ₙ-PepXₙ] polypeptide units in said polypeptide, with n being an integer of from 1 to 12; which encompasses n being 1, and
“PepCt” consists of a polypeptide having an amino acid length of 5 amino acid residues and located at the C-terminal end of the polypeptide of formula (III).

In these embodiments wherein “PepX₁” to “PepXₙ” each consists of, one independently from the other, a spacer polypeptide having an amino acid length of 0 amino acid residue, “[(I)ₙ-PepXₙ]” may also be represented by the formula “[(I)ₙ]” and the polypeptide of formula (III) may also be represented by the following formula (VII):

NH₂-PepNt-[(I)ₙ]ₙ-PepCt-COOH (VII),

with the variable parameters as defined above.

In a still preferred embodiment, the said antigenic polypeptide is represented by a polypeptide of the following formula (III):

NH₂-PepNt-[(I)ₙ-PepXₙ]ₙ-PepCt-COOH (III),

wherein:
“PepNt” consists of a polypeptide having an amino acid length of 5 amino acid residues and located at the N-terminal end of the polypeptide of formula (III);
“[(I)ₙ-PepXₙ]” consists of a polypeptide unit wherein:
   “(I)₁” to −“(I)ₙ” each consists of, one independently from each other, a polypeptide of formula “SWSNKS” (SEQ ID No 9), with n being an integer of 1; and
   “PepX₁” to “PepXₙ” each consists of, one independently from the other, a spacer polypeptide having an amino acid length of 0 amino acid residues, with n being an integer of from 1 to 12, which encompasses n being 1;
n is the number of [(I)ₙ-PepXₙ] polypeptide units in said polypeptide, with n being an integer of from 1 to 12, which encompasses n being 1; and
“PepCt” consists of a polypeptide having an amino acid length of 5 amino acid residues and located at the C-terminal end of the polypeptide of formula (III).

In these embodiments wherein “PepX₁” to “PepXₙ” each consists of, one independently from the other, a spacer polypeptide having an amino acid length of 0 amino acid residue, “[(I)ₙ-PepXₙ]” may also be represented by the formula “[(I)ₙ]” and the polypeptide of formula (III) may also be represented by the following formula (VII):

NH₂-PepNt-[(I)ₙ]ₙ-PepCt-COOH (VII),

with the variable parameters as defined above.

In a still preferred embodiment, the said antigenic polypeptide is represented by a polypeptide of the following formula (III):

NH₂-PepNt-[(I)ₙ-PepXₙ]ₙ-PepCt-COOH (III),

wherein:
“PepNt” consists of a polypeptide having an amino acid length of 6 amino acid residues and located at the N-terminal end of the polypeptide of formula (III);
“[(I)ₙ-PepXₙ]” consists of a polypeptide unit wherein:
   “(I)₁” to −“(I).” each consists of, one independently from each other, a polypeptide of formula “SWSNKS” (SEQ ID No 9), with n being an integer of 1; and
   “PepX₁” to “PepXₙ” each consists of, one independently from the other, a spacer polypeptide having an amino acid length of 0 amino acid residues, with n being an integer of from 1 to 12, which encompasses n being 1;
n is the number of [(I)ₙ-PepXₙ] polypeptide units in said polypeptide, with n being an integer of from 1 to 12, which encompasses n being 1; and
“PepCt” consists of a polypeptide having an amino acid length of 5 amino acid residues and located at the C-terminal end of the polypeptide of formula (III).

In these embodiments wherein “PepX₁” to “PepXₙ” each consists of, one independently from the other, a spacer polypeptide having an amino acid length of 0 amino acid residue, “[(I)ₙ-PepXₙ]” may also be represented by the formula “[(I)ₙ]” and the polypeptide of formula (III) may also be represented by the following formula (VII): NH₂-PepNt-[(I)ₙ]ₙ-PepCt-COOH (VII), with the variable parameters as defined above.

Thus, in certain embodiments of a peptide of formula (III) or (VII), PeptNt consists of a polypeptide having an amino acid length ranging from 4 to 6 amino acid residues and located at the N-terminal end of the polypeptide of formula (III).

In some preferred embodiments of a peptide of formula (III) or (VII), PepCt is a polypeptide comprising, or alternatively consisting of, the following amino acid sequence LDDIW (VI).

In some preferred embodiments, an antigenic polypeptide of formula (III) comprises, or alternatively consists of, the following amino acid sequence (SEQ ID No 5):

PWASNASWSNKSLDDIW (II).

In further preferred embodiments, an antigenic polypeptide of formula (III) comprises, or alternatively consists of, the following amino acid sequence (SEQ ID No 6):

PWNASWSNKSLDDIW (IV).

In still further preferred embodiments, an antigenic polypeptide of formula (III) comprises, or alternatively consists of, the following amino acid sequence (SEQ ID No 7):

CPWNASWSNKSLDDIW (V).

An antigenic polypeptide as described herein can be covalently linked through an amino acid residue to a carrier protein or a synthetic polymer.

In order to enhance peptide immunogenicity, the peptide of formula (I) can be covalently linked (“conjugated”) to a larger molecule, which serves as a carrier.

Attachment of the peptide to the carrier can be by one of several methods, including linking through a peptide Lys using glutaraldehyde (Reichlin, Methods Enzymol. 70: 159-165, 1980) or DCC procedures (for example, Atassi et al., Biochem. Biophys. Acta 670: 300-302, 1981), through a Peptide Asp or Glu using DCC (Bauminger et al., Methods Enzymol 70: 151-159, 1980), through a peptide Tyr using bis-diazotized benzidine (Walter et al., Proc. Nat. Acad. Sci. USA 77: 5197-5200, 1980), through photochemical attachment sites (Parker et al., Cold Spring Harbor Symposium—Modern Approaches to Vaccines, Ed. Chanock & Lerner, Cold Spring Harbor Press, New York, 1984), or through a peptide Cys (Liu et al., Biochem. 18: 690-697, 1979).

Peptide carrier conjugates can be separated from excess free peptide by dialysis or gel filtration. The level of loading of the peptide on the carrier can be determined either using a radioactive tracer to establish the loading level in a particular procedure, or by quantitative amino acid analysis of the conjugate, in comparison with the unloaded carrier. It is convenient, when using the latter technique, to incorporate a unique non-natural amino acid into the peptide, at the N-terminal or C-terminal side, such as Nle, which can then serve as a quantitative marker for peptide incorporation, as measured by amino acid analysis of the conjugate. This Nle can also function as a spacer between the antigenic site and any amino acid incorporated to facilitate attachment, such as Cys, Lys, or Tyr, as described above.

Preferably, said carrier protein is selected from the group consisting of keyhole limpet hemocyanin (KLH), bovine serum albumin, or diphtheria toxoid.

In a vaccine composition according to the invention, said synthetic polymer can be a multiple branch peptide construction comprising a core matrix comprised of lysine residues.

Radially branched systems using lysine skeletons in polymers have been used by J. P. Tam [Proc. Natl. Acad. Sci. U.S.A., 85, 5409-5413 (1988)] to develop antigens without the use of carriers. Those antigens were designed to generate vaccines against a variety of diseases. Specifically, antigens for generating vaccines against HIV infection are described by Tam in PCT patent application ser. no. WO93/03766, and in U.S. Pat. No. 5,229,490.

The core matrix is preferably a dendritic polymer which is branched in nature, preferably with each of the branches thereof being identical. The core matrix is based on a core molecule which has at least two functional groups to which molecular branches having terminal functional groups are covalently bonded. Exemplary for use to form the core matrix is lysine. A central lysine residue is bonded to two lysine residues, each through its carboxyl group, to one of the amino groups of the central lysine residue. This provides a molecule with four amino groups, which may be a core matrix for a structure comprising four polypeptides of formula (I). The manufacture of the above structures has been known in the art. See, e.g., Tam et al., J. Immun. 148, 914-920 (1992) and Wang et al., Science, 254, 285-288 (1991).

Additionally, spacers between said polypeptide and said carrier protein or synthetic polymer can be added. A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The invention concerns also a vaccine composition comprising a polypeptide comprising the amino acid sequence SWSNKS, said polypeptide, being covalently linked through an amino acid residue to a carrier protein or to a synthetic polymer.

Preferably, said carrier protein is selected from the group consisting of keyhole limpet hemocyanin (KLH), bovine serum albumin, or diphtheria toxoid.

The synthetic polymer can be a multiple branch peptide construction comprising a core matrix comprised of lysine residues. Spacers between said polypeptide and said carrier protein or synthetic polymer can be introduced.

Preferably, in the vaccine composition cited immediately above, there are spacers between said polypeptide and said carrier protein or synthetic polymer.

This invention is also directed to the use of an antibody directed against a polypeptide of formula (I) for manufacturing a pharmaceutical composition for treating a disease linked to the infection of an individual with a virus of the HIV family.

Additional Compounds

In a preferred embodiment, the vaccine composition according to the invention, further comprises one or more components selected from the group consisting of surfactants, absorption promoters, water absorbing polymers, substances which inhibit enzymatic degradation, alcohol, organic solvents, oils, pH controlling agents, preservatives, osmotic pressure controlling agents, propellants, water and mixture thereof.

Examples of appropriate supplementary carriers include, but are not limited to, sterile water, saline, buffers, phosphate-buffered saline, buffered sodium chloride, vegetable oils, Minimum Essential Medium (MEM), MEM with HEPES buffer, etc.

Optionally, the vaccine composition of the invention may contain conventional, secondary adjuvants in varying amounts depending on the adjuvant and the desired result. The customary amount ranges from about 0.02% to about 20% by weight, depending upon the other ingredients and desired effect.

Examples of suitable secondary adjuvants include, but are not limited to, stabilizers; emulsifiers; aluminium hydroxide; aluminium phosphate; pH adjusters such as sodium hydroxide, hydrochloric acid, etc.; surfactants such as Tween® 80 (polysorbate 80, commercially available from Sigma Chemical Co., St. Louis, Mo.); liposomes; iscom adjuvant; synthetic glycopeptides such as muramyl dipeptides; extenders such as dextran or dextran combinations, for example, with aluminium phosphate; carboxypolymethylene; bacterial cell walls such as mycobacterial cell wall extract; their derivatives such as Corynebacterium parvum; Propionibacterium acne; Mycobacterium bovis, for example, Bovine Calmette Guerin (BCG); vaccinia or animal poxvirus proteins; subviral particle adjuvants such as orbivirus; cholera toxin; N,N-dioctadecyl-N′,N′-bis(2-hydroxyethyl)-propanediamine (avridine); monophosphoryl lipid A; dimethyldioctadecylammonium bromide (DDA, commercially available from Kodak, Rochester, N.Y.); synthetics and mixtures thereof. Desirably, aluminium hydroxide is admixed with other secondary adjuvants or an immunoadjuvant such as Quil A.

Examples of suitable stabilizers include, but are not limited to, sucrose, gelatin, peptone, digested protein extracts such as NZ-Amine or NZ-Amine AS. Examples of emulsifiers include, but are not limited to, mineral oil, vegetable oil, peanut oil and other standard, metabolizable, non-toxic oils useful for injectable or intranasal vaccines compositions.

These adjuvants are identified herein as “secondary” merely to contrast with the above-described immunoadjuvant compounds.

Conventional preservatives can be added to the vaccine composition in effective amounts ranging from about 0.0001% to about 0.1% by weight. Depending on the preservative employed in the formulation, amounts below or above this range may be useful. Typical preservatives include, for example, potassium sorbate, sodium metabisulfite, phenol, methyl paraben, propyl paraben, thimerosal, etc.

The choice of inactivated, modified or other type of vaccine composition and method of preparation of the improved vaccine composition formulation of the present invention are known or readily determined by those of ordinary skill in the art.

When the polypeptide or formula (I) is not covalently bound to an immunoadjuvant, in the vaccine composition according to the invention, a pharmacologically effective amount of the immunoadjuvant compound described above may be given, for example orally, parenterally or otherwise, concurrently with, sequentially to or shortly after the administration of the polypeptide of formula (I).

As a general rule, the vaccine composition of the present invention is conveniently administered orally, parenterally (subcutaneously, intramuscularly, intravenously, intradermally or intraperitoneally), intrabuccally, intranasally, or transdermally.

### In Vitro Screening and Diagnosis Methods According to the Invention

### a) Screening Methods

The inventors have surprisingly found that a specific polypeptide, derived from the gp41 protein of HIV, markedly enhances the expression of the Nkp44L protein on CD4⁺ T-cells surface.

The invention also concerns a first method for the in vitro screening of compounds for preventing or treating a disease linked with the infection of an individual with an HIV virus, comprising the steps of:
(i) incubating a candidate compound to be tested with a polypeptide of formula (I),
(ii) assaying for the binding of the candidate compound to be tested with a polypeptide of formula (I).

The binding of the candidate compound to the polypeptide of formula (I) can be carried on by the one skilled in the art, for example by using a Two-hybrid system. Other means, known from the one skilled in the art can be used for the binding assays such as the use of bio sensor techniques (Edwards and Leatherbarrow (1997) or also by Szabo et al. (1995)), affinity chromatography, or High Throughput Screening (HTS), (Leblanc et al 2002).

The candidate compounds, which may be screened according to the screening method above, may be of any kind, including, without being limited to, natural or synthetic compounds or molecules of biological origin such as polypeptides.

Preferably, step (ii) consists of subjecting to a gel migration assay the mixture obtained at the end of step (i) and detecting the complexes formed between the candidate compound and the polypeptide of formula (I).

The gel migration assay can be carried out as known by the one skilled in the art.

The detection of the complexes formed between the complexes formed between the candidate compound and the polypeptide according to the invention can be easily observed by determining the stain position (protein bands) corresponding to the proteins analysed since the apparent molecular weight of a protein changes if it is part of a complex with another protein.

On one hand, the stains (protein bands) corresponding to the proteins submitted to the gel migration assay can be detected by specific antibodies for example antibodies specifically directed against a polypeptide of formula (I). One the other hand, a polypeptide of formula (I) can be tagged for an easier detection of the protein/candidate compound on the gel. For example, the polypeptide according to the invention can be fused to GST, HA, a poly-Histidine chain, or other detectable molecules in order to facilitate the identification of the different proteins on the gel.

The invention further concerns a second method for the in vitro screening of compounds for preventing or treating a disease linked to the infection of an individual with an HIV virus, comprising the steps of:
a) (i) bringing into contact a first CD4+ T-cell culture with a candidate compound, and HIV virus;
   (ii) bringing into contact a second CD4+ T-cell culture with HIV virus, in the absence of said candidate compound; and
b) detecting the presence of NKp44L at the CD4+ T-cells surface issued from the culture (i) and (ii).

The detection of the presence of NKp44L at the CD4+ T-cells surface can be carried out as known by the one skilled in the art, for instance by a cytofluorometric analysis as it is described in the part Material and methods, corresponding to the example 6.

Preferably, the method described above, comprises an additional step (c) which consists of selecting positively the candidate compound as a therapeutical agent when the level of expression of NKp44L at the CD4+ T-cells surface issued from the culture (ii) is higher than the level of expression of NKp44L at the CD4+ T-cells surface issued from the culture (i).

The comparison of the level of expression of NKp44L at the CD4+ T-cells surface can be assessed by counting the number of CD4+ T cells expressing NKp44L on their surface, using a fluorescence activated cell sorter (FACS), as described in the corresponding Material and Methods section.

Alternatively, the detection of the presence of NKp44L at the CD4+ T-cells surface can be carried out indirectly, by measuring the NK lysis activity of CD4+ T cells, as it is described in the section Material and Methods.

This particular embodiment of the step (b) of the screening method above, despite it consists of an in vitro method, has the technical advantage to directly reflect the therapeutical potential of the candidate compound by directly evidencing the biological activity of said candidate compound, as regards preventing the CD4⁺ T-cells cytolysis by the activated NK cells.

The activated NK cells may consist of cells from a NK cell line, such as the NK92 cell line described by Gong et al. (1994) or may consist of a primary culture of normal human purified NK cells.

The CD4⁺ T-cells that express the NKp44L protein may consist of CD4⁺ T-cells, eventually under the form of a cell line, that have been transfected with a vector that allow the expression by said cells of the NKp44L protein, or may consist of CD4⁺ T-cells that were initially purified from a blood sample of an HIV-infected patient.

In a specific embodiment of the screening method above, the activated NK cells and the CD4⁺ T-cells are autologous in that they both come from the same HIV-infected patient.

Preferably, the cytolysis measure consists of the conventional technique wherein the CD4⁺ T-cells, which are the target cells, are initially rendered radioactive with ⁵¹Cr, and wherein the cytolysis value consists of the percentage of cell lysis, as measured by the amount of ⁵¹Cr that is released in the cell culture medium by the lysed CD4⁺ T-cells.

Most preferably, the cytolysis value is obtained by assaying the cytolytic activity of the NK cells at increasing effector (NK cells) to target (CD4⁺ T-cells) ratios, for example from 1:1 to 50:1 effector: target cell ratios.

Candidate compounds for use in the screening methods described immediately above can be selected among the candidate compounds which bind to one or several polypeptides of formula (I).

Accordingly, the invention also concerns a method for the in vitro screening of compounds for preventing or treating a disease linked with the infection of an individual with an HIV virus, comprising the steps of:
(i) submitting a candidate compound to the first screening method above, and
(ii) submitting a candidate compound positively selected at step (i) to the second screening method described immediately above.

### b) Diagnosis Methods of the Invention

The inventors have found that, that antibodies directed against polypeptide of formula (I) are produced during HIV infection.

It has also been found according to the invention a statistically significant correlation between the expression level of antibodies directed against polypeptide of formula (I) collected from HIV-infected individuals and the level of CD4+ T cells.

It has been found according to the invention that the level of these antibodies decrease during the progression of HIV infection, especially as regards the development of the patient's immunodeficiency caused by the progressive depletion of his CD4+ T cells.

Besides, the inventors have found that this kind of kinetics is specific for antibodies directed against polypeptide of formula (I) and is not observed with antibodies directed against peptides T20 or T21 derived from HR1 and HR2 domains of gp41.

It flows from the experimental results obtained by the inventors which are briefly described above that the level of antibodies directed against a polypeptide of formula (I), contained in the serum of an individual, reveals itself to consist of an accurate biological marker of the progression status of the infection of an individual with an HIV virus. Further, the expression level said antibodies consists of a novel biological marker of the state of advancement of the HIV infection endowed with a very high biological significance, since it has been shown by the inventors that the level of antibodies directed against polypeptide of formula (I) is more important at the beginning of infection of a patient by HIV.

Accordingly, the invention also concerns a method for the in vitro assessment of the progression status of the infection of an individual with an HIV virus, wherein said method comprises the step of detecting in a sample from said individual, antibodies directed against a polypeptide of formula (I).

As used herein an “HIV” virus consists of either an HIV-1 or an HIV-2 virus, and more particularly any virus strain or isolate of an HIV-1 or an HIV-2 virus.

As used herein, the “assessment of the progression status” of the infection consists of raw experimental data indicative of the immunological status of the HIV-infected patient tested; Hence, as already mentioned above, there is a statistically relevant correlation between the amount of antibodies directed against the polypeptide of formula (I) and the CD4+ T cells count.

The sole detection of antibodies directed against a polypeptide of formula (I) might not be sufficient for a global accurate clinical diagnosis, or prognosis, of the progression status of the disease within the patient tested. Thus, the detection of antibodies directed against a polypeptide of formula (I) might be completed by, or combined with, other diagnosis or prognosis markers of the disease, for example one of the prior art markers that have previously been cited in the present specification.

The detection of antibodies directed against a polypeptide of formula (I) can be achieved using known techniques such as ELISA or RIA tests.

### Methods of Treatment According to the Invention

This invention also deals with a method for preventing or for treating a disease linked to the infection of an individual with a virus of the HIV family, wherein said method comprises a step of administering to a patient in need of such treatment an effective amount of an immunogenic or a vaccine composition comprising a polypeptide of formula (I) in combination with an immunoadjuvant compound.

The present invention further relates to a method for treating an HIV-infected patient, comprising administering to an individual in need thereof an antigenic polypeptide of the following formula (III):

NH₂-PepNt-[(I)ₙ-PepXₙ]ₙ-PepCt-COOH (III),

wherein:
“PepNt” consists of a polypeptide having an amino acid length from 2 to 10 amino acid residues and located at the N-terminal end of the polypeptide of formula (III);
“[(I)ₙ-PepXₙ]” consists of a polypeptide unit wherein:
   “(I)₁” to −“(I)ₙ” each consists of, one independently from each other, a polypeptide of formula “SWSNKS” (SEQ ID No 9), with n being an integer of 1; and
   “PepX₁” to “PepXₙ” each consists of, one independently from the other, a spacer polypeptide having an amino acid length of 0 amino acid residues, with n being an integer of from 1 to 12, which encompasses n being 1;
n is the number of [(I)ₙ-PepXₙ] polypeptide units in said polypeptide, with n being an integer of from 1 to 12, which encompasses n being 1; and
“PepCt” consists of a polypeptide having an amino acid length from 2 to 10 amino acid residues and located at the C-terminal end of the polypeptide of formula (III), wherein the said antigenic polypeptide raises an antibody response that does not neutralize the HIV virus and that inhibits the cytotoxicity of NK cells towards CD4+ T cells in patients infected by HIV.

In these embodiments wherein “PepX₁” to “PepXₙ” each consists of, one independently from the other, a spacer polypeptide having an amino acid length of 0 amino acid residue, “[(I)ₙ-PepXₙ]” may also be represented by the formula “[(I)ₙ]” and the polypeptide of formula (III) may also be represented by the following formula (VII):

NH₂-PepNt-[(I)ₙ]ₙ-PepCt-COOH (VII),

with the variable parameters as defined above.

Neutralization of a virus by a neutralizing antibody refers to the loss of infectivity of the virus upon interaction of said antibody and said virus.

In further embodiments, the invention relates to a method for treating an HIV-infected patient, comprising administering to an individual in need thereof an antigenic polypeptide of the following formula (III):

NH₂-PepNt-[(I)ₙ-PepXₙ]ₙ-PepCt-COOH (III),

wherein:
“PepNt” consists of a polypeptide having an amino acid length of 4 amino acid residues and located at the N-terminal end of the polypeptide of formula (III);
“[(I)ₙ-PepXₙ]” consists of a polypeptide unit wherein:
   “(I)₁” to −“(I)ₙ” each consists of, one independently from each other, a polypeptide of formula “SWSNKS” (SEQ ID No 9), with n being an integer of 1; and
   “PepX₁” to “PepXₙ” each consists of, one independently from the other, a spacer polypeptide having an amino acid length of 0 amino acid residues, with n being an integer of from 1 to 12, which encompasses n being 1;
n is the number of [(I)ₙ-PepXₙ] polypeptide units in said polypeptide, with n being an integer of from 1 to 12, which encompasses n being 1; and
“PepCt” consists of a polypeptide having an amino acid length of 5 amino acid residues and located at the C-terminal end of the polypeptide of formula (III),
   wherein said antigenic polypeptide is raising an antibody response that does not neutralize the HIV virus.

In these embodiments wherein “PepX₁” to “PepXₙ” each consists of, one independently from the other, a spacer polypeptide having an amino acid length of 0 amino acid residue, “[(I)ₙ-PepXₙ]” may also be represented by the formula “[(I)ₙ]” and the polypeptide of formula (III) may also be represented by the following formula (VII):

NH₂-PepNt-[(I)ₙ]ₙ-PepCt-COOH (VII),

with the variable parameters as defined above.

In still further embodiments, the invention relates to a method for treating an HIV-infected patient, comprising administering to an individual in need thereof of an antigenic polypeptide of the following formula (III):

NH₂-PepNt-[(I)ₙ-PepXₙ]ₙ-PepCt-COOH (III),

wherein:
“PepNt” consists of a polypeptide having an amino acid length of 5 amino acid residues and located at the N-terminal end of the polypeptide of formula (III);
“[(I)ₙ-PepXₙ]” consists of a polypeptide unit wherein:
   “(I)₁” to −“(I).” each consists of, one independently from each other, a polypeptide of formula “SWSNKS” (SEQ ID No 9), with n being an integer of 1; and
   “PepX₁” to “PepXₙ” each consists of, one independently from the other, a spacer polypeptide having an amino acid length of 0 amino acid residues, with n being an integer of from 1 to 12, which encompasses n being 1;
n is the number of [(I)ₙ-PepXₙ] polypeptide units in said polypeptide, with n being an integer of from 1 to 12, which encompasses n being 1; and
“PepCt” consists of a polypeptide having an amino acid length of 5 amino acid residues and located at the C-terminal end of the polypeptide of formula (III).

In these embodiments wherein “PepX₁” to “PepXₙ” each consists of, one independently from the other, a spacer polypeptide having an amino acid length of 0 amino acid residue, “[(I)ₙ-PepXₙ]” may also be represented by the formula “[(I)ₙ]” and the polypeptide of formula (III) may also be represented by the following formula (VII):

NH₂-PepNt-[(I)ₙ]ₙ-PepCt-COOH (VII),

with the variable parameters as defined above.

In yet further embodiments, the invention relates to a method for treating an HIV-infected patient, comprising administering to the said patient an antigenic polypeptide of the following formula (III):

NH₂-PepNt-[(I)ₙ-PepXₙ]ₙ-PepCt-COOH (III),

wherein:
“PepNt” consists of a polypeptide having an amino acid length of 6 amino acid residues and located at the N-terminal end of the polypeptide of formula (III);
“[(I)ₙ-PepXₙ]” consists of a polypeptide unit wherein:
   “(I)₁” to −“(I)ₙ” each consists of, one independently from each other, a polypeptide of formula “SWSNKS” (SEQ ID No 9), with n being an integer of 1; and
   “PepX₁” to “PepXₙ” each consists of, one independently from the other, a spacer polypeptide having an amino acid length of 0 amino acid residues, with n being an integer of from 1 to 12, which encompasses n being 1;
n is the number of [(I)ₙ-PepXₙ] polypeptide units in said polypeptide, with n being an integer of from 1 to 12, which encompasses n being 1; and
“PepCt” consists of a polypeptide having an amino acid length of 5 amino acid residues and located at the C-terminal end of the polypeptide of formula (III).

In these embodiments wherein “PepX₁” to “PepXₙ” each consists of, one independently from the other, a spacer polypeptide having an amino acid length of 0 amino acid residue, “[(I)ₙ-PepXₙ]” may also be represented by the formula “[(I)ₙ]” and the polypeptide of formula (III) may also be represented by the following formula (VII):

NH₂-PepNt-[(I)ₙ]ₙ-PepCt-COOH (VII),

with the variable parameters as defined above.

Thus, in certain embodiments of a peptide of formula (III) or (VII) that is used in a method for treating an HIV-infected patient, PeptNt consists of a polypeptide having an amino acid length ranging from 4 to 6 amino acid residues and located at the N-terminal end of the polypeptide of formula (III).

In some preferred embodiments of a peptide of formula (III) or (VII) that is used in a method for treating an HIV-infected patient, PepCt is a polypeptide comprising, or alternatively consisting of, the following amino acid sequence (SEQ ID No 8): LDDIW (VI).

In certain preferred embodiments, the invention relates to a method for treating an HIV infected patient, comprising administering to the said patient an antigenic polypeptide comprising, or alternatively consisting of, the following amino acid sequence (SEQ ID No 5):

PWASNASWSNKSLDDIW (II).

In further preferred embodiments, the invention relates to a method for treating an HIV infected patient, comprising administering to the said patient an antigenic polypeptide comprising, or alternatively consisting of, the following amino acid sequence (SEQ ID No 6):

PWNASWSNKSLDDIW (IV).

In still further preferred embodiments, the invention relates to a method for treating an HIV infected patient, comprising administering to the said patient an antigenic polypeptide comprising, or alternatively consisting of, the following amino acid sequence (SEQ ID No 7):

CPWNASWSNKSLDDIW (V).

It is disclosed in the art that a variety of immunogenic peptides derived from the HIV-1 gp41 protein, collectively encompassing most of the gp41 protein, would be able to generate antisera that neutralize HIV virus (Gilbert et al., U.S. Pat. No. 4,943,627).

More precisely, Gilbert et al. disclose the selection of peptides in a 425-751 Env region of HIV-1 gp41. It is reminded herein that the HIV-1 gp41 protein is of about 860 amino acid residues in length. In other words, the gp41 region, which is spanned by Gilbert et al. almost encompasses half of the whole gp41 protein. Gilbert et al. disclose a series of peptides derived from the HIV-1 gp41 protein. This series of peptides notably encompasses a region of Env between about amino acid 600 and amino acid 750 (4 peptides) and a region of gp41 between about amino acid 420 and amino acid 450 (1 peptide).

More precisely, Gilbert et al. have synthesized five gp41-derived peptides, namely: (i) peptide 1; amino acids 616-632 (of sequence PWNASWSNKSLEQIWNN; SEQ ID No 10), (ii) peptide 2: amino acids 667-680 (of sequence LLELDKWASLWNWF; SEQ ID No 11), (iii) peptide 3: amino acids 702-715 (of sequence LRIVFAVLSVVNRV; SEQ ID No 12), (iv) peptide 4: amino acids 728-751 (of sequence LPIPRGPDRPEGIEEEGGERDRDR; SEQ ID No 13) and (v) peptide 5: amino acids 426-450 (RIKQIINMWQEVGKAMYAPPISGQI; SEQ ID No 14).

According to Gilbert et al., the in vitro experimental data tend to show that antisera directed against each of the peptides 1-5 binds to the corresponding peptide; binds to the HIV virus; inhibits virus adsorption to cells as well as virus-mediated syncytium formation; and neutralizes the HIV virus.

Most importantly, antisera against peptides 1-5 would prevent HIV infection. More precisely, Gilbert et al. stated that the in vitro neutralizing activities of these anti-peptide antibodies indicate that the peptides 1-5 and antisera thereto are useful in vaccines for preventing HIV infection.

Thus, Gilbert et al. suggest that all gp41 peptides derived from most of the Env protein are functionally equivalents in that they are all able to raise antisera which neutralize the HIV virus and thus prevent HIV infection.

Moreover, by blocking the HIV virus from entering the host's sensitive cells, antisera directed against each of peptides 1-5 eventually compromises the subsequent virus-mediated physiological events that would have occurred if HIV virus would have maintained its infectivity, notably would have concurred to CD4 depletion.

In summary, Gilbert et al. teach to the one skilled in the art that almost every peptide derived from half of the gp41 protein (from about amino acid 420 to about amino acid 750) would successfully prevent HIV infection.

Surprisingly, in contrast to what was stated by Gilbert et al., peptides according to the present invention, deriving from a very discrete region of the HIV virus gp41 protein, are not able to neutralize the HIV virus (see Example 7). Therefore, HIV virus maintains its infectivity properties, and the antisera raised against the immunogenic peptides according to the invention result in non-neutralizing antibodies that counteract the subsequent physiological events following virus infection.

The object of the present invention was further confirmed by the results obtained by Vieillard et al. (2006, AIDS, Vol. 20: 1795-1804) which relate to the incidence of an antibody response against gp41 on the CD4⁺ T cell sensitivity to NK cell lysis.

The in vivo data provided by Vieillard et al. (2006) clearly show that the one skilled in the art would not even have selected the gp41-derived peptides disclosed by Gilbert for raising an antibody response that does not neutralize the HIV virus and that inhibits the cytotoxicity of NK cells towards CD4+ T cells in patients infected by HIV.

FIG. 3 of the article from Vieillard et al. (2006) illustrates a study of a possible correlation between (i) the presence of serum antibodies against various gp41 amino acid regions and (ii) CD4+ T cell count in HIV-infected individuals. Vieillard et al. (2006) have notably explored the effect of the presence of antibodies directed against various amino acid regions encompassed by the 525-750 amino acid region of gp41.

Peptides from the gp41 protein containing (i) the N-terminal domain (from amino acid 525 to amino acid 539, see FIG. 3a); (ii) the T21 motif (from amino acid 581 to amino acid 595, see FIG. 3b); (iii) the immune-dominant region (from amino acid 593 to amino acid 607, see FIG. 3c); (iv) the 3S motif (from amino acid 609 to amino acid 623, see FIG. 3d); (v) the gp41-T20 motif (from amino acid 658 to amino acid 671, see FIG. 3e); and (vi) the transmembrane region (from amino acid 689 to amino acid 703, see FIG. 3f) were used by Vieillard et al. (2006).

The results of FIG. 3 of Vieillard et al. (2006) clearly show that the sole antibodies able to actually reduce the HIV virus-induced CD4⁺ T cell depletion are those directed against the peptide containing the 3S motif, a peptide comprising the motif “SWSNKS” (SEQ ID No 9), i.e. a peptide of formula (III) or (VII) as described herein ((high statistical value—see graph (d) of FIG. 3). In contrast, the presence of antibodies directed against other peptides, like those illustrated in graphs (a), (b), (c), (e) and (f) of FIG. 3, exert strictly no effect on the HIV virus-induced CD4⁺ T cell depletion, or even increase immunosuppression (see peptide 593-607, FIG. 3, graph (c)).

Notably, antibodies directed against the peptides containing the gp41-T20 motif or the transmembrane region, encompassed within the gp41 regions from amino acid 600 to amino acid 750, as suggested by the teachings of Gilbert et al., have no effect on the HIV virus-induced CD4⁺ T cell depletion.

Thus, contrary to what is alleged in Gilbert et al., none of the peptides deriving from the 425-750 amino acid region of the gp41 protein, and more particularly from the 600-750 amino acid region of the gp41 protein, excepted a peptide of formula (III) or (VII) as described herein, is recognized by antibodies that neutralize the HIV virus and thus affect the biological consequences of the HIV infection.

Further, the results of FIG. 3 of Vieillard et al. (2006) clearly corroborate that most of the gp41-derived peptides tested are not endowed with an ability to counteract the NK cell-mediated CD4 T cell lysis occurring in HIV-infected individuals, excepted the peptides of formula (III) or (VII). that are described herein, which encompasses the peptides of SEQ ID N° 6 and SEQ ID No 7.

Most importantly, Vieillard et al. (2006) recall the previous unsuccessful attempts of a plurality of research teams to raise immune responses leading to a HIV neutralization (see the bridging paragraph from page 1802, right column, to page 1803, left column of Vieillard et al., 2006).

In other words, when referring to previous works relating to the use of gp41-derived peptides as antigens, which previous works encompass at least implicitly the experimental matter disclosed in Gilbert et al., Vieillard et al. (2006) fully confirm that such gp41-derived peptides are unable to raise the production of neutralizing antibodies, i.e. antibodies that prevent or inhibit HIV infection.

Within the scope of the present invention, neutralization of a HIV virus by an antibody may be performed by (i) inhibiting HIV virus binding to a target cell; (ii) inhibiting HIV virus uptake by a target cell; (iii) inhibiting HIV virus replication; (iv) inhibiting HIV virus particles release from infected cells.

A skilled in the art possesses the ability to perform any assay to assess neutralization of HIV virus. Notably, the neutralizing properties of antibodies may be assessed by a variety of tests, which all may assess the consequences of (i) inhibition of HIV virus binding to a target cell; (ii) inhibition of HIV virus uptake by a target cell; (iii) inhibition of HIV virus replication; (iv) inhibition of HIV virus particles release from infected cells. In other words, implementing different tests may lead to the observation of the same consequence, i.e. the loss of infectivity of the HIV virus.

For example, neutralization may be assessed by assessing the loss of infectivity of the virus upon interaction of said antibody and said virus.

For example, such assay may comprise the steps of:
a) incubating a HIV virus sample with an antibody to be tested;
b) incubating the mix from step a) with HIV-sensitive target cells bearing a reporter system, which expression is inducible upon HIV uptake into the target cells;
c) detecting and/or quantifying the level of expression of the reporting system.

Neutralizing antibodies may induce the expression of the reporting system, while a non-neutralizing antibody may not.

Within the scope of the present invention, HIV-sensitive target cells are selected in a group comprising naturally occurring CD4+ cells and cells genetically modified to express CD4 protein at their membrane surface.

In a preferred embodiment, HIV-sensitive target cells may bear reporter system, which expression is inducible upon HIV uptake into the target cells, hence, may be any HIV sensitive cells comprising a gene fusion comprising a HIV-inducible promoter in frame with a gene encoding an easily detectable protein.

In one embodiment, the HIV-inducible promoter is a TAT-inducible promoter, for example the HIV-1/LTR promoter (Long Terminal Repeat).

Easy detection and/or quantification of a protein may be performed by detecting and/or quantifying bioluminescence or enzymatic activity.

Any gene encoding a luminescent protein or any gene encoding a protein presenting a detectable enzymatic activity is suitable to be implemented within a neutralization assay.

In one embodiment, the gene encoding an easily detectable protein is a gene encoding a protein selected in the non-limited group comprising a galactosidase, an alkaline phosphatase, a chloramphenicol acetyltransferase, a luciferase, a fluorescent protein (green fluorescent protein (GFP), yellow fluorescent protein (YFP), red fluorescent protein (RFP), blue fluorescent protein (BFP).

In one embodiment, the HIV-sensitive target cells bearing a reporting system inducible upon HIV uptake into the target cells may be HeLa-P4C5 cells. HeLa-P4C5 cells are genetically modified HeLa cells that express CD4 and CCR5 and carry an integrated HIV/LTR-lacZ gene fusion, which is activated by TAT protein upon infection (Verrier et al., 1997).

In another embodiment, neutralization is assessed by assessing inhibition of HIV virus uptake by a target cell, comprising the steps of:
a) incubating a HIV virus sample with an antibody to be tested;
b) incubating the mix from step a) with isolated HIV-sensitive target cells;
c) assaying the survival of the HIV-sensitive target cells or assaying Plaque Forming Units (PFU).

Again, a neutralizing antibody may result in a survival of isolated HIV-sensitive target cells or a reduction of Plaque Forming Units, whereas a non-neutralizing antibody may not. Survival of HIV-1 sensitive cells may be assessed by evaluating the presence or the absence of cell lysis, consequently to the interaction with the virus in the presence of the antibodies to be tested. Neutralizing antibodies may prevent cell lysis, whereas non neutralizing antibodies may not. Cell lysis analysis may be easily performed by microscopy.

Thus, a peptide of formula (III) as described herein induces the production of antibodies directed against the said peptide and that are unable to neutralize an HIV-1 virus, when using the HIV neutralisation assays described above.

In a specific embodiment of the invention, said method of treatment comprises the following steps:
preparing a purified batch (HPLC, >95%) of polypeptide comprising the amino acid sequence of formula (II), linked to diphteria toxoid,
injecting diphteria toxoid alone to a first “control” group of mammals,
injecting the polypeptide comprising the amino acid sequence of formula (II), linked to diphteria toxoid, to a second “test” group of mammal.

Preferably, when the mammals used are of the Macaca mulatta species, they received 0.5 mg per mammal, and per injection of diphteria toxoid, linked or not, to the polypeptide cited above, followed by 3 new injections 3, 6 or 9 weeks later, in combination with Freund's incomplete adjuvant,
infecting the two groups of mammals with SHIV33, two weeks after the last injection, with 1 ml of a dilution at 50 TCID50/ml.
collecting samples consisting of serum and PBMC (peripheral blood mononuclear cells) at day 0, and 1 week after each injection (before infection) and then every week after infection by SHIV,
analysing the collected samples.

These analyses can comprises, without limitation, the control of the general aspect of the sample, the measurement of the amount of CD4+ T cells using a FACS, the measurement of the viral load by RT-PCR, the determination of the seroconversion by detecting antibodies directed against Env protein by EIA technique, the detection of antibodies directed against polypeptide of formula (II) by ELISA.

These analyses can comprise also the study of the effects of antibodies produced by said mammals on cells infected by HIV, and for example chronically infected cells. These cells are for example, U2 cells infected by HIV-sf2, Jurkat cells infected by HIV-BRU, or CD4+ T cells infected by BRU or collected from HIV infected patients.

Further, these analyses can comprise the measurement of NKp44L expression level, by FACS, the analysis of P24 amount, by EIA, or the analysis of NK cytotoxic activity of several strains of NK cells, such as autologous or allogenic NK92, NK3.3, or NKL cells, using a ⁵¹Cr test as described below.

A further object of the invention consists of a method for preventing or for treating a disease linked to the infection of an individual with a virus of the HIV family, wherein said method comprises a step of administering to a patient in need of such treatment an effective amount of an antibody directed against the polypeptide of formula (I).

The invention concerns also the use of a ligand compound which specifically binds to the polypeptide of formula (I), for manufacturing a pharmaceutical composition for preventing or treating a disease linked to the infection of an individual with a virus of the HIV family.

The invention also deals with the use of a polypeptide of formula (I), for manufacturing a vaccine composition for treating a disease linked to the infection of an individual with a virus of the HIV family.

The present invention is further illustrated by, without in any way being limited to, the following examples.

### EXAMPLES

### A. General Material and Methods

A.1 HIV-1 Infected Donors.

Blood samples of 25 HIV-1-infected patients were obtained from consenting donors at Hôpital Pitié-Salpétrière. Bio-clinical examinations included routine determinations of the viral load, total blood and CD4⁺ T lymphocyte counts.

As control group, Blood samples from 20 uninfected donors were obtained by leukapheresis from the blood bank (Hôpital Pitié-Salpétrière).

A.2 Cytofluorometric Analysis

A three-colors FACS analysis was performed on freshly harvested PBMC. Isotype-matched immunoglobulin served as the negative control (BD). Cells were incubated 1 h at 4° C., with the appropriate cocktail of antibodies. Anti-CD3; anti-CD4; anti-CD8, anti-CD56, anti-NKp44, anti-NKp46 or anti-NKp44L mAb. Erythrocytes were lysed using the FACS lysing solution (BD). A minimum of 20,000 leucocytes was analyzed on a FACScan, as previously described.

To measure the expression of cell surface activation markers, PBMC were stained with PE- or FITC-conjugated anti-HLA-DR, anti-CD69, anti-CD25, or anti-CD71 (all from BD) and analyzed by FACS.

A.3 Purification of T CD4⁺ Cells Expressing NKp44L

CD4⁺ T cell subset sorting was performed using the RosetteSepCD4⁺ enrichment kit (StemCell). CD4⁺ T expressing NKp44L were positively selected by a two-step magnetic separation, CD4⁺ T cells were incubated with 10 μg/ml of anti-NKp44L for 1-h at RT, followed by treatment with goat anti IgM mouse-coated Dynabeads (Dynal) at a bead-to-cell ratio of 10:1 for 30 min at RT. The cell fraction purity was determined by FACS analysis.

A.4 Isolation of Primary NK Cells and NK Cytotoxicity Assays.

NK lines were generated from PBMC, and then purified using the StemSep cell separation system and the NK cell enrichment antibody cocktail (StemCell technologies). NK purified cells were cultured in MyeloCult H5100 medium (StemCell technologies) supplemented with 100 units rhlL-2 (Boheringer). The purity of these preparations was evaluated by flow cytometry after staining with anti-CD3 (BD), anti-CD56 (BD), anti-NKp44, and anti-NKp46 mAbs.

The cytolytic activity was assayed in 4-h ⁵¹Cr-release assay as previously described. Briefly, the target cells were labeled for 2-h at 37° C. with 100 μCi per 10⁶ cells Na⁵¹Cr (Amersham), and washed twice with culture medium. The target cells were then distributed in round-bottomed 96-well microtiter plates (4×10³ cells per well), and the effector cells were added at several E/T ratio. The plates were incubated 4-h at 37° C. The supernatant were then collected and ⁵¹Cr-release was measured in a gamma counted. In experiments in which Abs were included, these were added to final concentration of 20 μg/ml. The relative specific ⁵¹Cr-release was calculated according to conventional methods. Values for spontaneous ⁵¹Cr-release, which are deducted in the calculation, were between 10 and 20% of the total incorporated radioactivity. The results are presented after subtraction of the nonspecific lysis obtained with control targets. Each point represents the average of triplicate values. The range of the triplicates was always within 5% of their mean.

A.5 Statistical Analysis

Correlation analyses were performed using Sperman's non-parametric rank correlation analysis. All calculations were performed using the GraphPad Prism.

### B. Material and Methods of the Examples 1-7

B.1 Purification of T CD4⁺ Cells

CD4⁺ T cell subset sorting was performed using the RosetteSepCD4⁺ enrichment kit (StemCell). The cell fraction purity was determined by FACS analysis.

B.2 Cytofluorometric Analysis

A two-colors FACS analysis was performed on purified CD4+ T cells. Isotype-matched immunoglobulin served as the negative control (BD). Cells were incubated 1 h at 4° C., with the appropriate cocktail of antibodies. anti-CD4 or anti-NKp44L mAb. A minimum of 20,000 CD4+ T cells was analyzed on a FACScan, as previously described. The intra-cellular expression of NKp44L was realized as previously described, briefly, the cells were incubated in 4% PFA buffer for 20 min, then washed in stained in presence of 0.1% saponin/PBS/1% BSA buffer at 4° C. the cells were then analyzed by FACS.

B.3 Isolation of Primary NK Cells and NK Cytotoxicity Assays.

NK lines were generated from PBMC, and then purified using the StemSep cell separation system and the NK cell enrichment antibody cocktail (StemCell technologies). NK purified cells were cultured in MyeloCult H5100 medium (StemCell technologies) supplemented with 100 units rhlL-2 (Boheringer). The purity of these preparations was evaluated by flow cytometry after staining with anti-CD3 (BD), anti-CD56 (BD), anti-NKp44, and anti-NKp46 mAbs.

The cytololytic activity was assayed in 4-h ⁵¹Cr-release assay as previously described. Briefly, the target cells were labeled for 2-h at 37° C. with 100 μCi per 10⁶ cells Na⁵¹Cr (Amersham), and washed twice with culture medium. The target cells were then distributed in round-bottomed 96-well microtiter plates (4×10³ cells per well), and the effector cells were added at several E/T ratio. The plates were incubated 4-h at 37° C. The supernatant were then collected and ⁵¹Cr-release was measured in a gamma counted. In experiments in which Abs were included, these were added to final concentration of 20 μg/ml. The relative specific ⁵¹Cr-release was calculated as previously described. Values for spontaneous ⁵¹Cr-release, which are deducted in the calculation, were between 10 and 20% of the total incorporated radioactivity. The results are presented after subtraction of the nonspecific lysis obtained with control targets. Each point represents the average of triplicate values. The range of the triplicates was always within 5% of their mean.

B.4 Recombinant Vaccinia Virus Expression HIV-1 Protein.

Purified CD4+ T cells were infected with wild type vaccinia virus (WT) or with the various recombinant vaccinia virus at a multiplicity infection of 20 PFU/cell were used as target cells. Recombinant vaccinia viruses for HIV-1-LAI Gag, Pol, Env, Nef, Tat and Vif proteins were provided by Transgene (Strasbourg, France).

B.5 Peptides and Pools of Peptides.

The synthetic 15-mers peptides were purchased from Epytop (Nîmes, France) or kindly provided by Agence Nationale de la Recherche sur le SIDA. All were more than 80% pure as shown by HPLC profiles. Pools of peptides included around 10 different peptides and each peptide overlap the previous continuous peptide for 11 residues.

B.6 mAbs Screened for their Capacity to Inhibit NK Lysis

Anti-NKp44 (44/8; IgG1) and anti-NKp44L mAb (#7.1; IgM) were obtained from 5-weekold BalB/c mice immunized with the ClonaCell-HY hybridoma cloning kit, according to the manufacturer's instructions (StemCell Technologies Inc.). The anti-mouse-peroxidase hybridoma screening reagent (Roche) and ELISA were used to select antibodies. Anti-NKp44 (44/8) mAb was prepared by immunizing the mice with NKp44-Ig protein, after specific deletion of the IgG1 human Fc fragment. To obtain #7.1 mAb (IgM), mice were immunized with acid-treated U2-sf2 cells. These cells were prepared as previously described (S. Sumitran-Karuppan, E. Moller, Transpl. Immunol. 4, 163, 1996.). Briefly, one million U2-Sf2 cells were washed 3 times in PBS and treated for 5 min on ice in an acid buffer prepared by mixing equal volumes of 0.263 M citric acid and 0.123 M Na2HPO4 containing 1% (w/v) BSA. After three more washes, the cells were resuspended in PBS and then irradiated and injected into the mice. All hybridomas were analyzed with 51Cr cytotoxic assays with untreated or acid-treated 721.221 cells as targets, and the specificity of the anti-NKp44 hybridoma was analyzed by ELISA with different fusion proteins. The #7.1 mAb (IgM) mAb was purified on a mannan-binding protein (MBP) column (Pierce), after ammonium sulfate precipitation (50% saturated solution); the anti-NKp44 mAb was purified on a protein A/G column (Pierce). The purity of each purified mAb was confirmed on SDS-PAGE.

B.7 Neutralizing Assays

a) Virus Preparation.

HIV-1 virus was obtained by standard techniques. Virus supernatants were frozen and stored at −80° C.
b) Peptides, Antibodies and ELISA.

Purified unconjugated and KLH-conjugated 3S peptide (Vieillard et al., 2005, Proc Natl Acad Sci USA, Vol. 102: 10981-10986) (purity of each peptide >95%) were purchased from Covalabs (Villeurbanne, France). Six Balb/c 4-6 month female mice had two subcutaneous injections of 20 μg of KLH-linked peptide in incomplete Freund's adjuvant, about two weeks apart. This study was carried out in strict accordance with the recommendations of the European guidelines for the care and use of laboratory animals (European directive 86/609/EEC, Nov. 24, 1986). All efforts were made to minimize suffering of animals. The protocol was approved by the local ethics committee (Authorization Number: 91-510). The sera were titrated by enzyme-linked immunosorbent assays (ELISA) that used Maxisorp plates (Nunc) coated with 100 ng 3S peptide overnight at 4° C., as described (Vieillard et al., 2006, AIDS, Vol. 20: 1795-1804). The purity of each purified Ig is ranged between 92 and 97%.

c) Neutralization Assays.

Polyclonal IgG fractions from mouse antisera were isolated with the Nab Protein A/G Spin kit and then desalted with Zeba spin desalting columns (both from Thermo Scientific), according to the manufacturer's instructions. Immunoglobulin fractions were concentrated with Centricon Centrifugal Filter Units (Millipore) and then quantified with the BCA protein assay kit (Thermo Scientific). Purified Igs were tested at starting concentrations of 20 μg/mL followed by five 2-fold dilutions. Neutralization of the purified Igs was tested with assays that used infectious viruses (Jacobs et al., 2005, J Biol Chem, Vol. 280: 27284-27288) for the ability of various purified Igs to inhibit HIV-1 entry from Tier 1 (BRU, NDK, MW965.26) and tier 2 (JR-CSF, YU-2, VI191, TV1) strains of different clades, or ROD HIV-2 was measured in HeLa-P4C5 reporter cells. Infectious replication-competent viruses (200TCID₅₀) were incubated with various concentrations of Igs for 30 min; target cells were then added. At 48 h after infection, β-galactosidase activity in HeLa-P4C5 lysates was measured with the CPRG β-galactosidase Kit. A cutoff value of 2.5 times background was applied to determine positive values in TCID assays. Data were expressed as IC₅₀.

### C. Results

### Example 1 Effects of Several HIV Viral Proteins on NKp44L Expression

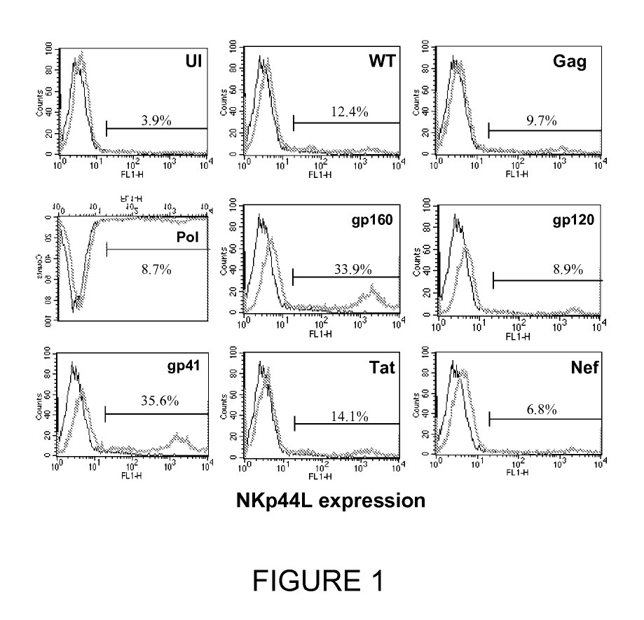

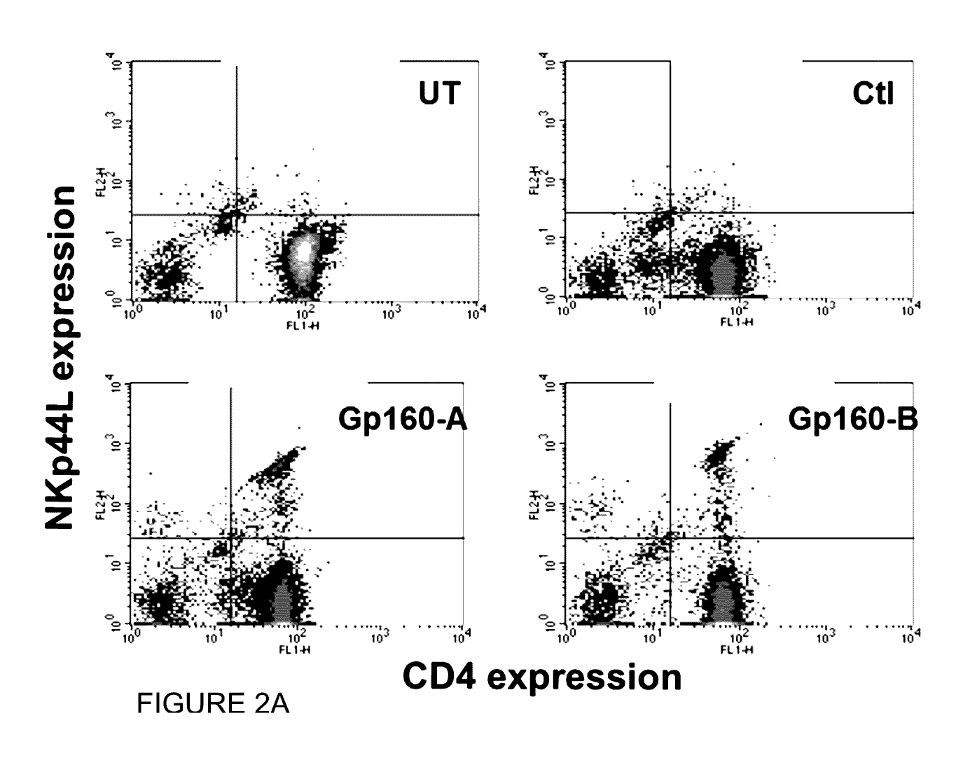

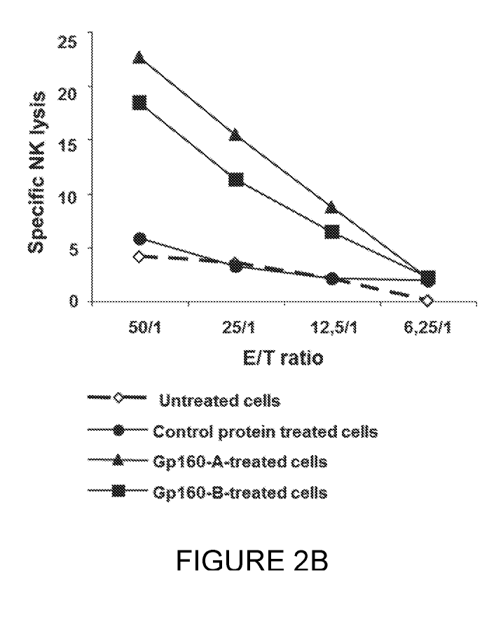

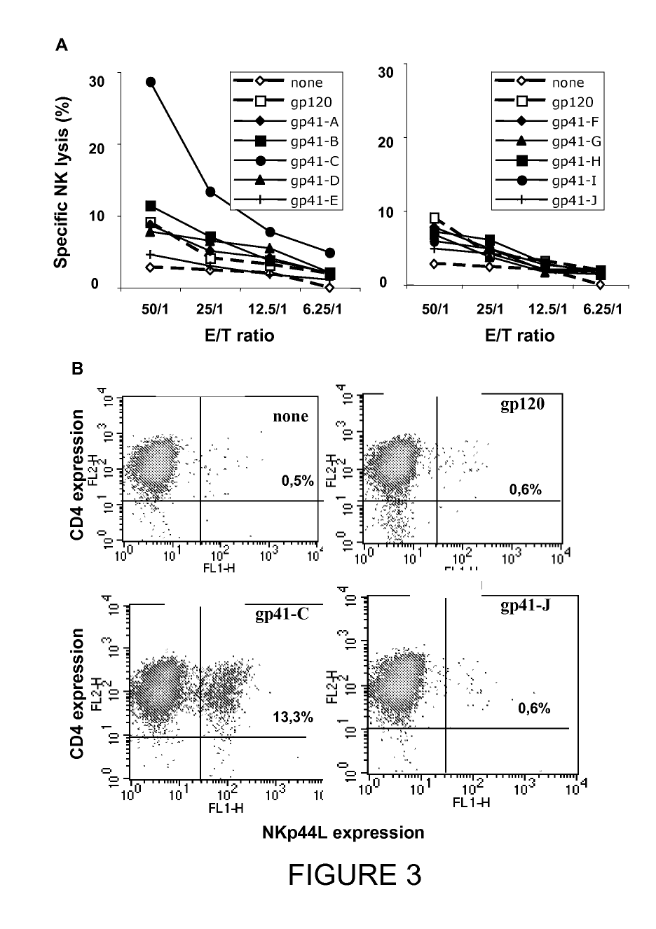

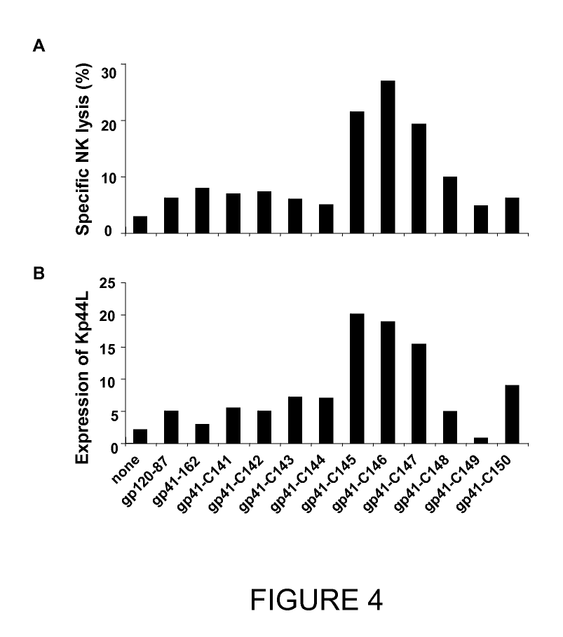

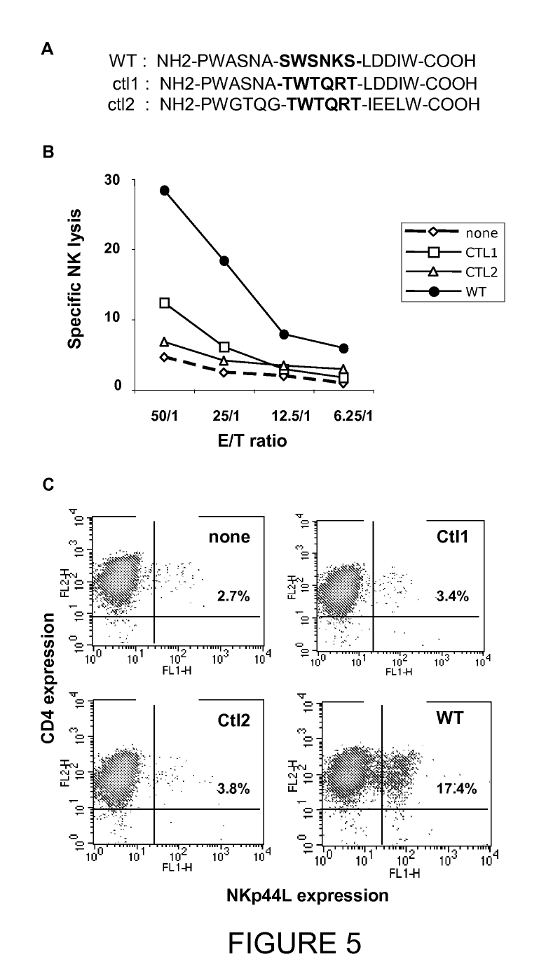

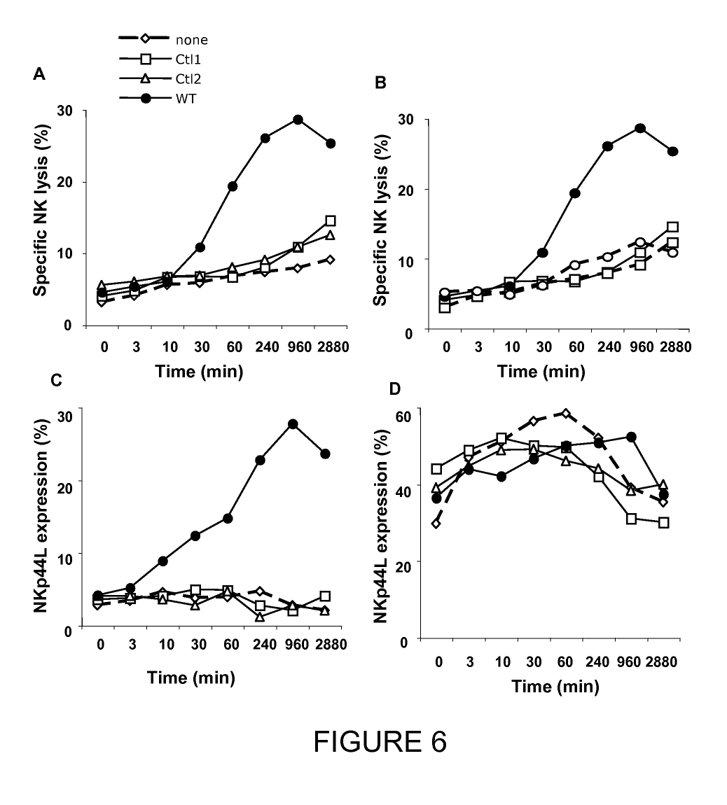

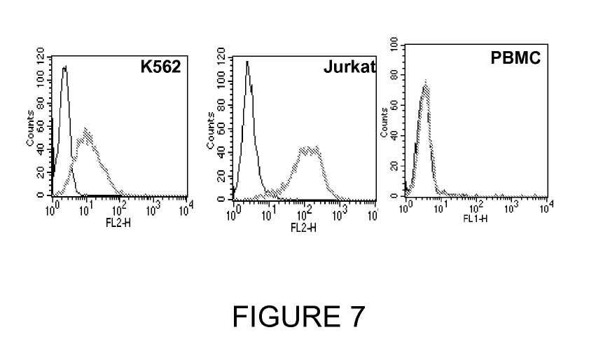

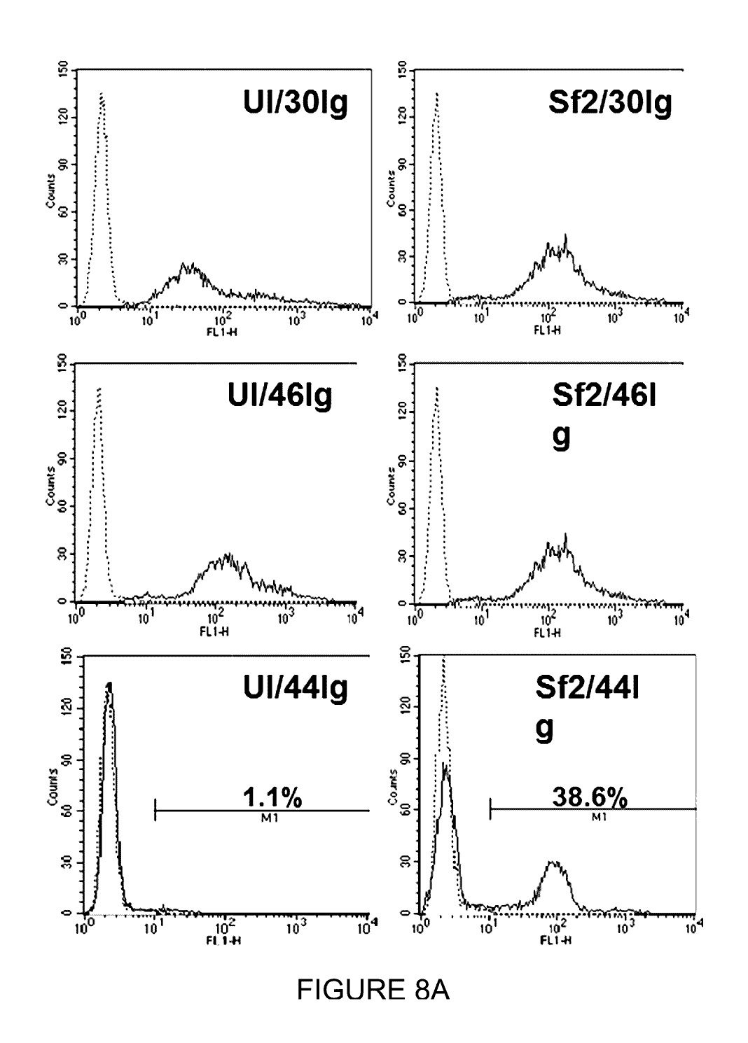

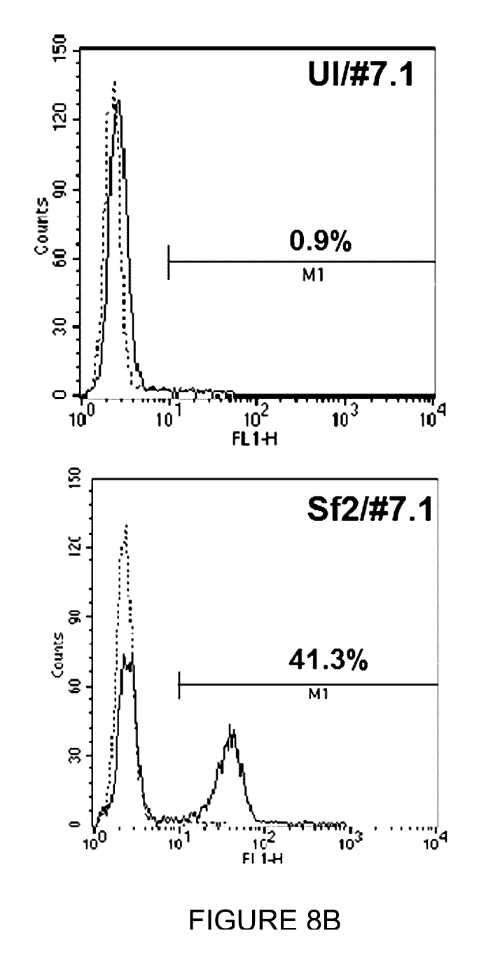

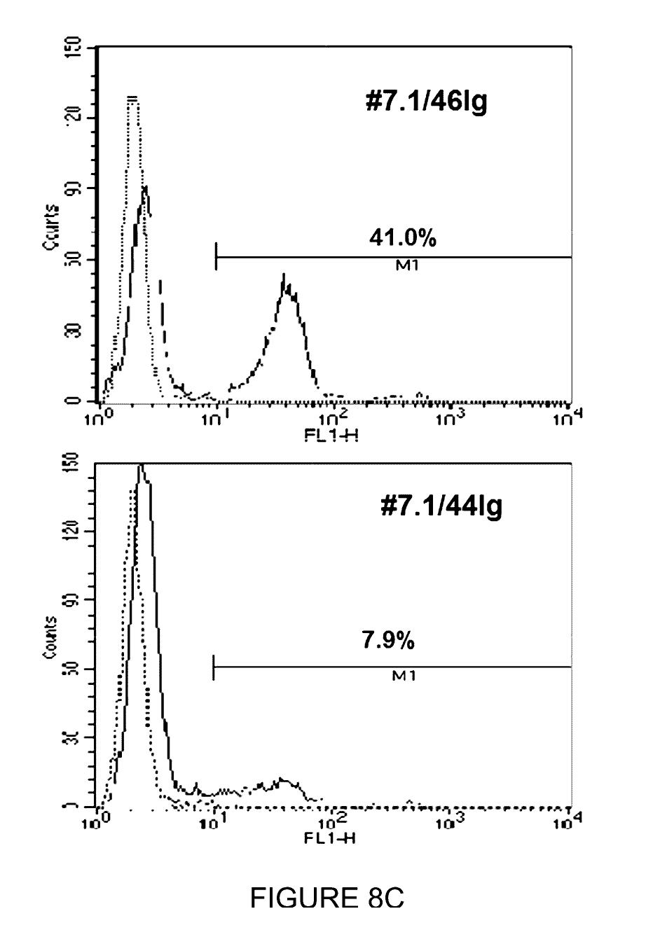

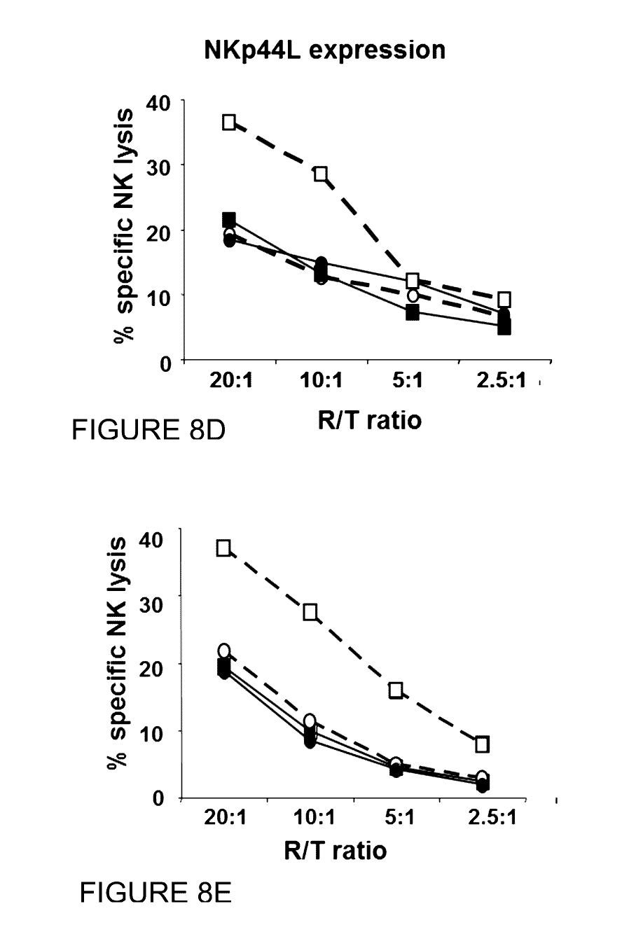

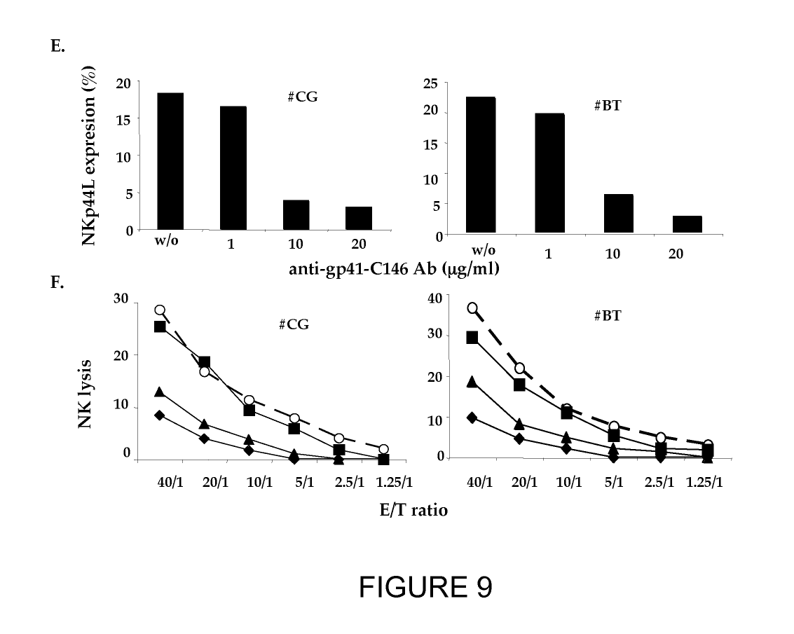

The effect of HIV viral protein on NKp44L expression was examined using infection with recombinant vaccinia virus expressing HIV viral protein. As shown in FIG. 1, the expression of NKp44L was markedly enhanced in CD4+ T cells infected with vaccinia virus expressing the gp160 (33.9%) or the gp41 HIV Env proteins (35.6%). In contrast, neither other HIV proteins tested, like Gag, Pol, Tat, nef, vif, nor gp120, influenced the cell surface expression of NKp44L protein. Furthermore, the role of the Env protein to enhance the expression of NKp44L was confirmed in a non-viral system. Purified CD4+ T cells were treated with recombinant gp160 protein provided of two different origins, and as shown in FIG. 2A, these gp160 recombinant proteins influenced the expression of NKp44L. Indeed, 10.7% and 9.6% of CD4+ T cells expressed NKp44L after treatment with the gp160-A and gp160-B, respectively. On the other hand, no effect was observed with untreated cells or cells incubated with a control protein. All together, these results show that the recombinant gp160 protein markedly enhances the cell surface expression of NKp44L on CD4+ T-cells surface.

### Example 2 gp160 Induces the NK Lysis of CD4+ T Cells

Comparison of NK lysis activity from the untreated cells, the cells treated with the control protein or the cells treated with the both recombinant gp160 proteins (FIG. 2B), shows that target cell lysis was increased in the presence of CD4+ T cells cultured with recombinant gp160 protein. The use of two different types of recombinant gp160 proteins indicates that the procedure used to induce over-expression of NKp44L and increased of NK lysis activity had no influence on the outcome of the experiments. Together, these results indicate that gp41 HIV Env protein was required for the over-expression of NKp44L correlated with a strong increased of NK lysis activity.

### Example 3 Identification of the Peptide Motif of the gp41 Env Protein Involved in the Increased of NK Lysis Activity

The effect of pool of overlap peptides prepared, as described in the Materials & Methods section, has been tested, to include all of the gp41 protein. CD4+ T cells were incubated with 5 μg of each pool of peptides and tested against activated NK cells. As shown in FIG. 3, NK lysis was increased in cells incubated with the pool of peptides named gp41C, but not with all of the other pool of peptides. The expression of NKp44L in purified CD4+ T cells treated with each of the pool of peptides has been tested. This receptor was only detectable in cells treated with pool gp41C, and the percentage of positive cells was 13.3%. In no instance, NKp44L was detected on the cells incubated with the other pool of peptides tested. This suggested that one or several peptide motifs included in the pool gp41C was directly implicated in the increased of NK lysis via the over-expression of NKp44L. Repeated experiments with all of the peptides included in the pool gp41C was then tested. As shown in FIG. 4A, the NK cytotoxic activity was strongly increased in presence of the peptides gp41-C145, gp41-C146, and gp41-C147. By contrast, with the other peptides tested, the NK lysis activity remained low, closed to the background. In parallel, the expression of NKp44L was increased after pretreatment of CD4+ T cells with the peptides gp41-C145, gp41-C146, and gp41-C147, with a percentage of positive cells varied between 22 and 16%, but not with the other peptides (less than 7% of positive cells) (FIG. 4B). These results indicated that a peptide specific to gp41 Env HIV protein could increase a NK lysis activity. Additional support to this conclusion come from that the continuous peptides named gp41-C145, gp41-C146, and gp41-C147 included a common peptide motif NH2-SWSNKS-COOH, This motif specific to the gp41 HIV-1 protein was strongly conserved. After having shown that some continuous peptides of the gp41 are some major mediators of NK lysis, it was important to assess if the peptide motif NH2-SWSNKS-COOH was directly implicated in the NK lysis of CD4+ T cells. Preliminary experiment with this 6-mers peptide shown any increased of cell surface expression of NKp44L or NK cytotoxic activity, suggesting that this sequence is too small or too rapidly attack by some peptidases. However, to test this hypothesis, two 15-mers peptides derived from gp41-C146 (WT) included some mutation inside the NH2-SWSNKS-COOH motif (Ctl1) or in all of the 15-mers sequence (Ctl2) have been constructed (FIG. 5A). As shown in FIG. 5B, the NK cytotoxic activity was strongly increased in presence of the peptide WT. By contrast, with untreated cells (none) or the treated with the both control peptides. Similar pattern was observed concerning the cell surface expression of NKp44L, indeed, in cells treated with the WT peptide, approximately 17.4% of CD4 T cells expressed this marker. By contrast, the percentage of NKp44L+ cells was less than 4% in untreated cells or cells treated by the control peptides. These results show that the NH2-SWSNKS-COOH motif included in the gp41 protein is strongly implicated in the NK lysis of CD4+ T cells.

The effect of gp41 peptide is time dependent (FIG. 6). NK lysis activity started after 30 min of incubation with the WT peptide and approached a maximum closed to 4-days. On the other hand, no significant effect is observed after treatment with the untreated cells or the cells treated with the control peptides. Furthermore, the increased of NK lysis activity is strongly inhibited after pretreatment of cells with anti-NKp44L mAb, confirming that the NK activity is directly correlated with an increase of cell surface expression of NKp44L, in cells treated with the WT peptide (FIG. 6C). However, kinetic study of the cell surface expression of NKp44L revealed that this receptor was rapidly expressed at the cell surface, indeed after 10 min of treatment with WT peptide, around 10% of CD4+ T cells expressed this protein, and the maximum of expression (approximately 30%) was obtained 4-days after treatment. The very fast cell surface expression of NKp44L suggested an absence of new synthesis of NKp44L, and suggested that this protein was present inside the CD4+ T cells cultured with IL2. This hypothesis was confirmed by an intracellular staining of NKp44L. As show in FIG. 6D, high expression of NKp44L was detectable inside the cells, and this independently of the presence of peptides.

### Example 4 Cell Surface Expression of NKp44L of Different Human Cells

As shown on FIG. 7, the surface expression of NKp44L on K562, Jurkat, and resting PBMC has been tested. The cells were incubated with 1 μg/ml of anti-NKp44L mAb anti-NKp44L mAb (grey thick line) or with the IgM isotype control (black thin), and analyzed by flow cytometry. It is clearly shown that, contrary to PBMC, tumor cells, like jurkat, and K562 cells express NKp44L on their surface.

### Example 5 Identification of #7.1, an Anti-NKp44L mAb that Specifically Inhibits NKp44-Mediated NK Lysis

To study the related function and expression of this ligand during HIV-1 infection, a library of mAbs screened for their capacity to inhibit NK lysis has been tested. One of them, the #7.1 mAb, revealed an epitope expressed on NKp44L. Specific staining with this mAb was found in HIV-infected U2 cells only, not in uninfected U2 cells (FIG. 8B), and it yielded a level of staining similar to that of the NKp44-Ig fusion protein (compare FIGS. 8A and 8B). Inhibition of the #7.1 mAb staining by this fusion protein confirmed that #7.1 mAb specifically interact with a NKp44 ligand (NKp44L) (FIG. 8C). A control experiment showed no effect with the NKp46-Ig fusion protein (FIG. 8C). Furthermore, when U2 cells chronically infected by the HIV-1 Sf2 strain were pre-treated with the #7.1 mAb, their NK-mediated lysis decreased sharply (as much as 40%) (FIG. 8D). Treatment of NK cells by the anti-NKp44 mAb produced the same effect (FIG. 8E). Note that in both series of experiments the magnitude of the lysis obtained in the presence of anti-NKp44 or 7.1 mAb was similar to that observed with uninfected cells. We obtained similar results with Jurkat cells chronically infected by the HIV-1 BRU strain (data not shown). Together, these data strongly suggest that NKp44L is expressed during HIV-1 infection and that #7.1 mAb reacts specifically with NKp44L on HIV-1 infected cells.

### Example 6 Critical Role of the NH2-SWSNKS-COOH Motif from the gp41 HIV Protein

A rabbit anti-gp41-C146 peptide polyclonal Ab has been tested for its capacity of inhibition of NKp44L expression and for sensitivity to NK lysis. Briefly, highly purified peptide (gp41-C146 peptide) (>95%) was linked to the KLH and injected in several rabbits. Serum titres were determined by ELISA on PeptiPlaks. Antibodies were purified by chromatography. NKp44L expression was substantially lower (7.8%) in purified CD4+ T cells from two HIV-1 infected patients that were incubated with purified anti-gp41-C146 polyclonal Ab than in purified and then untreated cells (27.2%) or in those treated with a control Ab (27.9%) (FIG. 9E). This effect was confirmed by the drastic inhibition of NK activity in the presence of the anti-gp41-C146 polyclonal Ab (FIG. 9F). These results strongly suggest that the gp41 NH2-SWSNKS-COOH motif plays a key role in inducing NKp44L expression during HIV infection and that the gp41 protein participates in the selective destruction of CD4+ T cells by activated NK cells.

### Example 7 Non Neutralizing Properties of Antibodies Raised Against a Peptide of Formula (VI)

The results are depicted in Table 1 hereunder.

TABLE 1
Lack of neutralizing activity for various strains of HIV-1 for infection
on HeLa P4C5 cells of murine Ig antibodies from immunized
mice with the NH₂-CPWNASWSNKSLDDIW-COOH
peptide (SEQ ID No 7).

| | | | Co-receptor | | IC₅₀ |
|---|---|---|---|---|---|
| Strain | Tier | Clade | specificity | | (μg/ml) |
| BRU | 1 | B | X4 | WT | >20 |
| NDK | 1 | B | X4 | WT | >20 |
| JR-CSF | 2 | B | R5 | WT | 18.1 |
| YU-2 | 2 | B | R5 | WT | >20 |

A good neutralizing activity would be for IC₅₀ values of less than 5 μg/ml. Consequently, the results of Table 1 show that antibodies raised against a peptide of formula (III) are unable to neutralize any of the HIV-1 strains tested.

### SEQUENCES DISCLOSED IN THE PRESENT INVENTION

| | Nature of the | |
|---|---|---|
| SEQ ID No | sequence | Description of the sequence |
| 1 | Protein | Sequence of the human NKp44L protein |
| 2 | Protein | Sequence of the human NKp44 protein |
| 3 | DNA | Sequence of the human NKp44L nucleic acid |
| 4 | DNA | Sequence of the human NKp44 nucleic acid |
| 5 | Peptide | Peptide of formula (II) |
| 6 | Peptide | Peptide of formula (IV) |
| 7 | Peptide | Peptide of formula (V) |
| 8 | Peptide | Peptide of formula (VI) |
| 9 | Peptide | Sequence of the “SWSNKS” motif |
| 10 | Peptide | Sequence of peptide 1 from Gilbert et al. |
| | | (amino acids 616-632 of gp41 of HIV virus) |
| 11 | Peptide | Sequence of peptide 2 from Gilbert et al. |
| | | (amino acids 667-680 of gp41 of HIV virus) |
| 12 | Peptide | Sequence of peptide 3 from Gilbert et al. |
| | | (amino acids 702-715 of gp41 of HIV virus) |
| 13 | Peptide | Sequence of peptide 4 from Gilbert et al. |
| | | (amino acids 728-751 of gp41 of HIV virus) |
| 14 | Peptide | Sequence of peptide 5 from Gilbert et al. |
| | | (amino acids 426-450 of gp41 of HIV virus) |

### REFERENCES

Ameisen and Capron, Cell dysfunction and depletion in AIDS: the programmed cell death hypothesis, Immunol. Today, 12(4):102-5, 1991.
Bagnarelli et al., Molecular profile of human immunodeficiency virus type 1 infection in symptomless patients and in patients with AIDS, J. Virol., 66(12):7328-35, 1992.
Cantoni et al., NKp44, a triggering receptor involved in tumor cell lysis by activated human natural killer cells, is a novel member of the immunoglobulin superfamily, J. Exp. Med., 189(5):787-96, 1999.
CDC, 1993 revised classification system for HIV infection and expanded surveillance case definition for AIDS among adolescents and adults, MMWR, 41:1-19, 1992.
CDC, Revision of the surveillance case definition of acquired immunodeficiency syndrome, MMWR, 36:3S-15S, 1987.
Clark et al., High titers of cytopathic virus in plasma of patients with symptomatic primary HIV-1 infection, N. Engl. J. Med., 324(14):954-60, 1991.
Coombs et al., Plasma viremia in human immunodeficiency virus infection, N. Engl. J. Med., 321(24):1626-31, 1989.
Edwards and Leatherbarrow, Determination of association rate constants by an optical biosensor using initial rate analysis, Analytical Biochemistry, 246:1-6, 1997.
Embretson et al., Massive covert infection of helper T lymphocytes and macrophages by HIV during the incubation period of AIDS, Nature, 362(6418):359-62, 1993.
Fauci, Multifactorial nature of human immunodeficiency virus disease: implications for therapy, Science, 262(3136):1011-8, 1993a.
Felici, Selection of antibody ligands from a large library of oligopeptides expressed on a multivalent exposition vector, J. Mol. Biol., 222:301-310, 1991.
Finkel et al., Apoptosis occurs predominantly in bystander cells and not in productively infected cells of HIV- and SIV-infected lymph nodes, Nature Medicine, 1(2):129-34, 1995.
Fox et al., Detection of HIV-1 RNA in the lamina propria of patients with AIDS and gastrointestinal disease, J. Infect. Dis., 159(3):467-71, 1989.
Garry, Potential mechanisms for the cytopathic properties of HIV, AIDS, 3(11):683-94, 1989.
Golding et al., Common epitope in human immunodeficiency virus I (HIV) I-GP41 and HLA class II elicits immunosuppressive antibodies capable of contributing to immune dysfunction in HIV-infected individuals, J. Clin. Invest., 83(4):1430-5, 1989.
Gong et al., Characterization of a human cell line (NK-92) with phenotypical and functional characteristics of activated natural killer cells, Leukemia, 8(4):652-658, 1994.
Ho et al., Quantitation of human immunodeficiency virus type 1 in the blood of infected persons, N. Engl. J. Med., 321(24):1621-5, 1989.
Ho et al., Rapid turnover of plasma virions and CD4 lymphocytes in HIV-1 infection, Nature, 373:123-6, 1995.
Hoxie et al., Alterations in T4 (CD4) protein and mRNA synthesis in cells infected with HIV, Science, 234(4780):1123-7, 1986.
Hugin et al., A virus-encoded superantigen in a retrovirus-induced immunodeficiency syndrome of mice, Science, 252(5004):424-7, 1991.
Janeway, Immune recognition. Mls: makes little sense, Nature, 349(6309):459-61, 1991.
Koenig et al., Group-specific, major histocompatibility complex class-I restricted cytotoxic responses to human immunodeficiency virus I (HIV-1) envelope proteins by cloned peripheral blood T cells from an HIV-1 infected individual, Proc. Natl. Acad. Sci. USA, 85(22):8638-42, 1988.
Koga et al., High levels of heterodisperse RNAs accumulate in T cells infected with human immunodeficiency virus and in normal thymocytes, Proc. Natl. Acad. Sci. USA, 85(12):4521-5, 1988.
Kohler and Milstein, Continuous cultures of fused cells secreting antibody of predefined specificity, Nature, 256:495, 1975.
Kozbor et al., A comparative analysis of the phenotypic characteristics of available fusion partners for the construction of human hybridomas, Hybridoma, 2(1):7-16, 1983.
Laurent-Crawford et al., The cytopathic effect of HIV is associated with apoptosis, Virology, 185(2):829-39, 1991.
Leblanc et al., Homogeneous time-resolved fluorescence assay for identifying p53 interactions with its protein partners, directly in a cellular extract, Anal. Biochem., 308(2):247-54, 2002.
Leger et al., Humanization of a mouse antibody against human alpha-4 integrin: a potential therapeutic for the treatment of multiple sclerosis, Hum. Antibodies, 8(1):3-16, 1997.
Leonard et al., Cytopathic effect of human immunodeficiency virus in T4 cells is linked to the last stage of virus infection, Proc. Natl. Acad. Sci. USA, 85(10):3570-4, 1988.
Lifson et al., AIDS retrovirus induced cytopathology: giant cell formation and involvement of CD4 antigen, Science, 232(4754):1123-7, 1986.
Lyerly et al., Human T-cell lymphotropic virus IIIB glycoprotein (gp120) bound to CD4 determinants on normal lymphocytes and expressed by infected cells serves as target for immune attack, Proc. Natl. Acad. Sci. USA, 84(13):4601-5, 1987.
Martineau et al., Expression of an antibody fragment at high levels in the bacterial cytoplasm, J. Mol. Biol., 280(1):117-127, 1998.
Michael et al., Viral DNA and mRNA correlate with the stage of human immunodeficiency virus (HIV) type 1 infection in humans: evidence for viral replication in all stages of HIV disease, J. Virol., 66(1):310-6, 1992.
Muro-Cacho et al., Analysis of apoptosis in lymph nodes of HIV-infected persons. Intensity of apoptosis correlates with the general state of activation of the lymphoid tissue and not with stage of disease or viral burden, J. Immunol., 154(10):5555-66, 1995.
Oldenburg et al., Proc. Natl. Acad. Sci. USA, 85(8):2444-2448, 1992.
Pantaleo and Fauci, Apoptosis in HIV infection, Nature Medicine, 1995b; 1(2):118-20, 1995b.
Pantaleo et al., HIV infection is active and progressive in lymphoid tissue during the clinically latent stage of disease, Nature, 362(6418):355-8, 1993b.
Pantaleo et al., AS. The immunopathogenesis of human immunodeficiency virus infection, N. Engl. J. Med., 328(5):327-35, 1993a.
Parmley and Smith, Antibody-selectable filamentous fd phage vectors: affinity purification of target genes, Gene, 73:305-318, 1988.
Pauza et al., Reinfection results in accumulation of unintegrated viral DNA in cytopathic and persistent human immunodeficiency virus type 1 infection of CEM cells, J. Exp. Med., 172(4):1035-42, 1990.
Piatak et al., High levels of HIV-1 in plasma during all stages of infection determined by competitive PCR, Science, 259(5102):1749-54, 1993.
Popovic et al., Detection, isolation and continuous production of cytopathic retroviruses (HTLV-III) from patients with AIDS and pre-AIDS, Science, 224(4648):497-500, 1984.
Reinmann et al., AIDS Res. Hum. Retroviruses, 13(11):933-943, 1997.
Ridder et al., Generation of rabbit monoclonal antibody fragments from a combinatorial phage display library and their production in the yeast Pichia pastoris, Biotechnology (N Y), 13(3):255-260, 1995.
Schnittman et al., Evidence for susceptibility of intrathymic T-cell precursors and their progeny carrying T-cell antigen receptor phenotypes TCR alpha beta+and TCR gamma delta+to human immunodeficiency virus infection: a mechanism for CD4+(T4) lymphocyte depletion, Proc. Natl. Acad. Sci. USA, 87(19):7727-31, 1990b.
Sodroski et al., Role of the HTLV-III/LAV envelope in syncytium formation and cytopathicity, Nature, 322(6078):470-4, 1986.
Stanley et al., CD34+ bone marrow cells are infected with HIV in a subset of seropositive individuals, J. Immunol., 149(2):689-97, 1992.
Szabo et al., Surface plasmon resonance and its use in biomolecular interaction analysis (BIA), Curr. Opinion Struct. Biol., 5(5):699-705, 1995.
Terai et al., Apoptosis as a mechanism of cell death in cultured T lymphoblasts acutely infected with HIV-1, J. Clin. Invest., 87(5):1710-5, 1991.
Valadon et al., Peptide libraries define the fine specificity of anti-polysaccharide antibodies to Cryptococcus neoformans, J. Mol. Biol., 261:11-22, 1996.
Verrier et al. Antibodies to several conformation-dependent epitopes of gp120/gp41 inhibit CCR-5-dependent cell-to-cell fusion mediated by the native envelope glycoprotein of a primary macrophage-tropic HIV-1 isolate. Proceedings of the National Academy of Sciences 94(17): 9326-9331, 1997.
Vieillard et al., Specific adaptive humoral response against a gp41 motif inhibits CD4 T-cell sensitivity to NK lysis during HIV-1 infection, AIDS Vol. 20(14): 1795-1804, 2006.
Vieillard et al., A vaccine strategy against AIDS: An HIV gp41 peptide immunization prevents NKp44L expression and CD4+ T cell depletion in SHIV-infected macaques, PNAS, Vol. 105(6): 2100-2104, 2008.
Vieillard et al., Specific anti-gp41 antibodies predict HIV-1 disease progression, JAIDS, Vol. 61(3): 403-405, 2012.
Vitale et al., NKp44, a novel triggering surface molecule specifically expressed by activated natural killer cells, is involved in non-major histocompatibility complex-restricted tumor cell lysis, J. Exp. Med., 187(12):2065-72, 1998
Wei et al., Viral dynamics in human immunodeficiency virus type 1 infection, Nature, 373:117-22, 1995.
Westerink et al., Peptide mimicry of the meningococcal group C capsular polysaccharide, Proc. Natl. Acad. Sci. USA, 92:4021-4025, 1995.
Zagury et al., Long-term cultures of HTLV-III infected T cells: a model of cytopathology of T-cell depletion in AIDS, Science, 231(4740):850-3, 1986.

1. A method for treating an HIV infected patient, comprising administering to an individual an effective amount of an antigenic polypeptide of the following formula (III):
NH₂-PepNt-[(I)ₙ-PepXₙ]ₙ-PepCt-COOH (III),
wherein:
“PepNt” consists of a polypeptide having an amino acid length from 4 to 6 amino acid residues and located at the N-terminal end of the polypeptide of formula (III);
“[(I)ₙ-PepXₙ]” consists of a polypeptide unit wherein:
“(I)₁” to −“(I)ₙ” each consists of, one independently from each other, a polypeptide of formula “SWSNKS”, with n being an integer of 1; and
“PepX₁” to “PepXₙ” each consists of, one independently from the other, a spacer polypeptide having an amino acid length of 0 amino acid residues, with n being an integer of from 1 to 12;
n is the number of [(I)ₙ-PepXₙ] polypeptide units in said polypeptide, with n being an integer of from 1 to 12; and
“PepCt” consists of a polypeptide having an amino acid length of 5 amino acid residues and located at the C-terminal end of the polypeptide of formula (III),
wherein the said antigenic polypeptide raises an antibody response that does not neutralize the HIV virus and that inhibits the cytotoxicity of NK cells towards CD4+ T cells in patients infected by HIV.

2. The method according to claim 1, wherein:
“PepNt” consists of a polypeptide having an amino acid length of 6 amino acid residues and located at the N-terminal end of the polypeptide of formula (III);
“[(I)ₙ-PepXₙ]” consists of a polypeptide unit wherein:
“(I)₁” to −“(I)ₙ” each consists of, one independently from each other, a polypeptide of formula “SWSNKS”, with n being an integer of 1; and
“PepX₁” to “PepXₙ” each consists of, one independently from the other, a spacer polypeptide having an amino acid length of 0 amino acid residues, with n being an integer of from 1 to 12, which encompasses n being 1;
n is the number of [(I)ₙ-PepXₙ] polypeptide units in said polypeptide, with n being an integer of from 1 to 12, which encompasses n being 1; and
“PepCt” consists of a polypeptide having an amino acid length of 5 amino acid residues and located at the C-terminal end of the polypeptide of formula (III).

3. The method of claim 2, wherein the antigenic compound of formula (III) is represented by a polypeptide comprising, or alternatively consisting of, the following amino acid sequence: PWASNASWSNKSLDDIW (II).

4. The method according to claim 1, wherein:
“PepNt” consists of a polypeptide having an amino acid length of 5 amino acid residues and located at the N-terminal end of the polypeptide of formula (III);
“[(I)ₙ-PepXₙ]” consists of a polypeptide unit wherein:
“(I)₁” to −“(I)ₙ” each consists of, one independently from each other, a polypeptide of formula “SWSNKS” (SEQ ID No 9), with n being an integer of 1; and
“PepX₁” to “PepXₙ” each consists of, one independently from the other, a spacer polypeptide having an amino acid length of 0 amino acid residues, with n being an integer of from 1 to 12, which encompasses n being 1;
n is the number of [(I)ₙ-PepXₙ] polypeptide units in said polypeptide, with n being an integer of from 1 to 12, which encompasses n being 1; and
“PepCt” consists of a polypeptide having an amino acid length of 5 amino acid residues and located at the C-terminal end of the polypeptide of formula (III).

5. The method according to claim 1, wherein:
“PepNt” consists of a polypeptide having an amino acid length of 4 amino acid residues and located at the N-terminal end of the polypeptide of formula (III);
“[(I)ₙ-PepXₙ]” consists of a polypeptide unit wherein:
“(I)₁” to −“(I)ₙ” each consists of, one independently from each other, a polypeptide of formula “SWSNKS”, with n being an integer of 1; and
“PepX₁” to “PepXₙ” each consists of, one independently from the other, a spacer polypeptide having an amino acid length of 0 amino acid residues, with n being an integer of from 1 to 12, which encompasses n being 1;
n is the number of [(I)ₙ-PepXₙ] polypeptide units in said polypeptide, with n being an integer of from 1 to 12, which encompasses n being 1; and
“PepCt” consists of a polypeptide having an amino acid length of 5 amino acid residues and located at the C-terminal end of the polypeptide of formula (III).

6. The method of claim 1, wherein the antigenic compound of formula (III) is conjugated to a carrier protein or to a synthetic polymer.

7. The method of claim 6, wherein the carrier protein is keyhole limpet hemocyanin (KLH), bovine serum albumin or diphteria toxoid.

8. The method of claim 6, wherein the synthetic polymer is a multiple branch peptide construction comprising a core matrix comprised of lysine residues.

9. The method of claim 6, wherein there is a spacer between the polypeptide and the carrier protein or synthetic polymer.

10. The method of claim 9, wherein the composition further comprises an immunoadjuvant compound.

11. The method of claim 10, wherein the immunoadjuvant compound is selected from the group consisting of Freund complete adjuvant, Freund incomplete adjuvant, aluminium hydroxide, calcium phosphate, aluminium phosphate, potassium phosphate, Cholera toxin (CT) and its B subunit (CTB), toxins from Bordetella pertusssis (PT), labile toxin (LT) from Escherichia coli, monophosphoryl lipid A, CpG oligonucleotides, imidazoquinolones, oil in water emulsions comprising squalene and synthetic copolymers, muramyl dipeptides and their derivatives, saponins and immunostimulating complexes (ISCOMs), and dimethyldioctadecylammonium bromide or chloride (DDA).

12. An antibody directed against an antigenic polypeptide of the following formula (III):
NH₂-PepNt-[(I)ₙ-PepXₙ]ₙ-PepCt-COOH (III),
wherein:
“PepNt” consists of a polypeptide having an amino acid length from 4 to 6 amino acid residues and located at the N-terminal end of the polypeptide of formula (III);
“[(I)ₙ-PepXₙ]” consists of a polypeptide unit wherein:
“(I)₁” to −“(I)ₙ” each consists of, one independently from each other, a polypeptide of formula “SWSNKS”, with n being an integer of 1; and
“PepX₁” to “PepXₙ” each consists of, one independently from the other, a spacer polypeptide having an amino acid length of 0 amino acid residues, with n being an integer of from 1 to 12;
n is the number of [(I)n-PepXₙ] polypeptide units in said polypeptide, with n being an integer of from 1 to 12; and
“PepCt” consists of a polypeptide having an amino acid length of 5 amino acid residues and located at the C-terminal end of the polypeptide of formula (III),
wherein the said antibody does not neutralize the HIV virus and inhibits the cytotoxicity of NK cells towards CD4+ T cells in patients infected by HIV.


SEQUENCE LISTING (14 sequences)

SEQ ID NO: 1 - protein, 1168 aa, Homo sapiens
MSIVIPLGVDTAETSYLEMAAGSEPESVEASPVVVEKSNSYPHQLYTSSSHHSHSYIGLPYADHNYGARPPPTPPASPPPSVLISKNEVGIFTTPNFDETSSATTISTSEDGSYGTDVTRCICGFTHDDGYMICCDKCSVWQHIDCMGIDRQHIPDTYLCERCQPRNLDKERAVLLQRRKRENMSDGDTSATESGDEVPVELYTAFQHTPTSITLTASRVSKVNDKRRKKSGEKEQHISKCKKAFREGSRKSSRVKGSAPEIDPSSDGSNFGWETKIKAWMDRYEEANNNQYSEGVQREAQRIALRLGNGNDKKEMNKSDLNTNNLLFKPPVESHIQKNKKILKSAKDLPPDALIIEYRGKFMLREQFEANGYFFKRPYPFVLFYSKFHGLEMCVDARTFGNEARFIRRSCTPNAEVRHEIQDGTIHLYIYSIHSIPKGTEITIAFDFDYGNCKYKVDCACLKENPECPVLKRSSESMENINSGYETRRKKGKKDEDISKEKDTQNQNITLDCEGATNKMKSPETKQRKLSPLRLSVSNNQEPDFIDDIEEKTPISNEVEMESEEQIAERKRKMTREERKMEAILQAFARLEKREKRREQALERISTAKTEVKTECKDTQIVSDAEVIQEQAKEENASKPTPAKVNRTKQRKSFSRSRTHIGQQRRRHRTVSMCSDIQPSSPDIEVTSQQNDIENTVLTIEPETETALAEIITETEVPALNKCPTKYPKTKKHLVNEWLSEKNEKTGKPSDGLSERPLRITTDPEVLATQLNSLPGLTYSPHVYSTPKHYIRFTSPFLSEKRRRKEPTENISGSCKKRWLKQALEEENSAILHRFNSPCQERSRSPAVNGENKSPLLLNDSCSLPDLTTPLKKRRFYQLLDSVYSETSTPTPSPYATPTHTDITPMDPSFATPPRIKSDDETCRNGYKPIYSPVTPVTPGTPGNTMHFENISSPESSPEIKRRTYSQEGYDRSSTMLTLGPFRNSNLTELGLQEIKTIGYTSPRSRTEVNRQCPGEKEPVSDLQLGLDAVEPTALHKTLETPAHDRAEPNSQLDSTHSGRGTMYSSWVKSPDRTGVNFSVNSNLRDLTPSHQLEVGGGFRISESKCLMQDDTRGMFMETTVFCTSEDGLVSGFGRTVNDNLIDGNCTPQNPPQKKKSPVGNFVGSNVV

SEQ ID NO: 2 - protein, 262 aa, Homo sapiens
MAWRALHHWLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTPWTPRDLVSSQTQTQSCVPPTAGARQAPESPSTIPVPSQPQNSTLRPGPAAPIALVPVFCGLLVAKSLVLSALLVWWGDIWWKTVMELRSLDTQKATCHLQQVTDLPWTSVSSPVEREILYHTVA

SEQ ID NO: 3 - DNA, 3507 bp, Homo sapiens
atgagcatagtgatcccattgggggttgatacagcagagacgtcatacttggaaatggctgcaggttcagaaccagaatccgtagaagctagccctgtggtagttgagaaatccaacagttatccccaccagttatataccagcagctcacatcattcacacagttacattggtttgccctatgcggaccataattatggtgctcgtcctcctccgacacctccggcttcccctcctccatcagtccttattagcaaaaatgaagtaggcatatttaccactcctaattttgatgaaacttccagtgctactacaatcagcacatctgaggatggaagttatggtactgatgtaaccaggtgcatatgtggttttacacatgatgatggatacatgatctgttgtgacaaatgcagcgtttggcaacatattgactgcatggggattgataggcagcatattcctgatacatatctatgtgaacgttgtcagcctaggaatttggataaagagagggcagtgctactacaacgccggaaaagggaaaatatgtcagatggtgataccagtgcaactgagagtggtgatgaggttcctgtggaattatatactgcatttcagcatactccaacatcaattactttaactgcttcaagagtttccaaagttaatgataaaagaaggaaaaaaagcggggagaaagaacaacacatttcaaaatgtaaaaaggcatttcgtgaaggatctaggaagtcatcaagagttaagggttcagctccagagattgatccttcatctgatggttcaaattttggatgggagacaaagatcaaagcatggatggatcgatatgaagaagcaaataacaaccagtatagtgagggtgttcagagggaggcacaaagaatagctctgagattaggcaatggaaatgacaaaaaagagatgaataaatccgatttgaataccaacaatttgctcttcaaacctcctgtagagagccatatacaaaagaataagaaaattcttaaatctgcaaaagatttgcctcctgatgcacttatcattgaatacagagggaagtttatgctgagagaacagtttgaagcaaatgggtatttctttaaaagaccatacccttttgtgttattctactctaaatttcatgggctagaaatgtgtgttgatgcaaggacttttgggaatgaggctcgattcatcaggcggtcttgtacacccaatgcagaggtgaggcatgaaattcaagatggaaccatacatctttatatttattctatacacagtattccaaagggaactgaaattactattgcctttgattttgactatggaaattgtaagtacaaggtggactgtgcatgcctcaaagaaaacccagagtgccctgttctaaaacgtagttctgaatccatggaaaatatcaatagtggttatgagaccagacggaaaaaaggaaaaaaagacgaagatatttcaaaagaaaaagatacacaaaatcagaatattactttggattgtgaaggagcgaccaacaaaatgaagagcccagaaactaaacaaagaaagctttctccactgagactatcagtatcaaataatcaggaaccagattttattgatgatatagaagaaaaaactcctattagtaatgaagtagaaatggaatcagaggagcagattgcagaaaggaaaaggaagatgacaagagaagaaagaaaaatggaagcaattttgcaagcttttgccagacttgaaaaaagagagaaaagaagagaacaagctttggaaaggatcagcacagccaaaactgaagttaaaactgaatgtaaagatacacagattgtcagtgatgctgaagttattcaggaacaagcaaaagaagaaaatgctagcaagccaacccctgccaaagtaaatagaactaaacagagaaaaagtttttctcggagtaggactcacattggacagcagcgtcggagacacagaactgtcagcatgtgttcagatatccagccatcttctcctgatatagaagttacttcacaacaaaatgatattgaaaatactgtacttacaatagaaccagaaactgaaactgcactagcagaaataattactgaaactgaagttccagcacttaataaatgtcctaccaagtaccccaaaacaaagaagcacttggttaatgaatggttaagtgagaagaatgagaagacaggaaaaccttcagatggcctttcagaaaggcctctacgcataactacagatcctgaagtgttagctacacaactcaattctttaccaggtctcacttacagcccccatgtatactccactcctaagcattatattagatttacttcaccattcctttcagaaaaaaggagaagaaaagaacctactgaaaacatttctggttcatgcaagaagcgatggttgaaacaagctctggaagaagaaaattcagcaattttacatagatttaattcaccctgtcaagaaagatccagaagtcctgcagtcaatggtgaaaataaaagtccactactattaaatgacagctgttcccttccagatttaactacaccactaaaaaaacgaagattttatcagttgctagattcggtttactcagaaacctccacacctactccttccccgtatgctacaccaactcacaccgatattactcctatggacccatcttttgccacgcctccacggataaaatcagatgatgaaacttgtagaaatggttataaacccatatattcaccagttaccccagtaactcctggtacaccaggaaataccatgcactttgagaatatttcttccccagaaagttctccagaaataaagagacgcacttatagtcaagagggatatgacagatcttcaaccatgttaacattggggccttttagaaattctaatttaactgaactgggtctgcaagaaataaagactattggttatacgagccctaggagtaggactgaagtcaacaggcagtgtcctggagaaaaggaacctgtgtcagaccttcagctaggactcgatgcagttgagccaactgccctacataaaaccctggaaacgcctgcacatgacagggctgagcccaacagccaactggactcgactcactctggacggggcacaatgtattcttcctgggtaaagagccctgacagaacaggagttaacttctcagtgaactccaacttgagggacctgacaccctcgcatcagttggaggttggaggaggcttccgaataagtgagtcaaagtgcctgatgcaggatgatactagaggcatgtttatggaaacaactgtgttttgtacttccgaagatgggcttgtatctggtttcggacggactgttaatgacaatttgatcgacgggaattgcacaccccagaatccaccacaaaagaaaaagagtccagttggcaactttgtgggaagcaatgtagtatag

SEQ ID NO: 4 - DNA, 787 bp, Homo sapiens
atggcctggcgagccctacaccactggctactgctgctgctgttcccaggctctcaggcacaatccaaggctcaggtacttcaaagtgtggcagggcagacgctaaccgtgagatgccagtacccgcccacgggcagtctctacgagaagaaaggctggtgtaaggaggcttcagcacttgtgtgcatcaggttagtcaccagctccaagcccaggacgatggcttggacctctcgattcacaatctgggacgaccctgatgctggcttcttcactgtcaccatgactgatctaagagaggaagactcaggacattactggtgtagaatctaccgcccttctgacaactctgtctctaagtccgtcagattctatctggtggtatctccagcctctgcctccacacagaccccctggactccccgcgacctggtctcttcacagacccagacccagagctgtgtgcctcccactgcaggagccagacaagcccctgagtctccatctaccatccctgtcccttctcagccacagaactccacgctccgccctggccctgcagcccccattgccctggtgcctgtgttctgtggactcctcgtagccaagagcctggtgctgtcagccctgctcgtctggtggggggacatatggtggaaaaccgtgatggagctcaggagcctggatacccaaaaagccacctgccaccttcaacaggtcacggaccttccctggacctcagtttcctcacctgtagagagagaaatattatatcacactgttgcaa

SEQ ID NO: 5 - protein, 17 aa, Artificial Sequence
  Synthetic Peptide
PWASNASWSNKSLDDIW

SEQ ID NO: 6 - protein, 15 aa, Artificial Sequence
  Synthetic Peptide
PWNASWSNKSLDDIW

SEQ ID NO: 7 - protein, 16 aa, Artificial Sequence
  Synthetic Peptide
CPWNASWSNKSLDDIW

SEQ ID NO: 8 - protein, 5 aa, Artificial Sequence
  Synthetic Peptide
LDDIW

SEQ ID NO: 9 - protein, 6 aa, Artificial Sequence
  Synthetic Peptide
SWSNKS

SEQ ID NO: 10 - protein, 17 aa, Artificial Sequence
  Synthetic Peptide
PWNASWSNKSLEQIWNN

SEQ ID NO: 11 - protein, 14 aa, Artificial Sequence
  Synthetic Peptide
LLELDKWASLWNWF

SEQ ID NO: 12 - protein, 14 aa, Artificial Sequence
  Synthetic Peptide
LRIVFAVLSVVNRV

SEQ ID NO: 13 - protein, 24 aa, Artificial Sequence
  Synthetic Peptide
LPIPRGPDRPEGIEEEGGERDRDR

SEQ ID NO: 14 - protein, 25 aa, Artificial Sequence
  Synthetic Peptide
RIKQIINMWQEVGKAMYAPPISGQI